(19) United States
(12) Patent Application Publication
(10) Pub. No.: US 2024/0050762 A1
(43) Pub. Date: Feb. 15, 2024

(54) ELECTROENCEPHALOGRAM (EEG) BASED TRANSCRANIAL MAGNETIC STIMULATION (TMS) THERAPIES

(71) Applicant: WAVE NEUROSCIENCE, INC., Newport Beach, CA (US)

(72) Inventors: James William PHILLIPS, Fountain Valley, CA (US); Alexander RING, Newport Beach, CA (US)

(73) Assignee: WAVE NEUROSCIENCE, INC., Newport Beach, CA (US)

(21) Appl. No.: 18/280,797

(22) PCT Filed: Mar. 25, 2022
(86) PCT No.: PCT/US22/71357

§ 371 Date: Sep. 7, 2023

Related U.S. Application Data
(60) Provisional application No. 63/166,944, filed on Mar. 26, 2021.

(51) Int. Cl. A61N 2/00; A61B 5/369

(52) U.S. Cl. CPC A61N 2/006; A61B 5/369

(57) ABSTRACT

A method for providing transcranial magnetic stimulation (TMS) based on electroencephalogram (EEG) data. The method may include use of a portable neuro-electroencephalogram synchronization therapy (NEST) system for capturing EEG data from a patient. The method may also include use of an electrophysiology database and customized TMS treatment system for receiving the EEG data from the portable NEST system. The electrophysiology database and customized TMS treatment system may determine a TMS treatment based on the received EEG data. The portable NEST system may deliver synchronized TMS based on the determined TMS treatment.

### CROSS-REFERENCE

This application claims the benefit of U.S. Provisional Application No. 63/166,944, filed Mar. 26, 2021, entitled METHODS, SYSTEMS, KITS AND APPARATUSES FOR PROVIDING TRANSCRANIAL MAGNETIC STIMULATION (TMS) BASED ON ELECTROENCEPHALOGRAM (EEG) DATA, which application is incorporated herein in its entirety by reference.

This application is related to the following co-pending patent applications: application Serial No. PCT/US22/71355 filed Mar. 25, 2022; and application Serial No. PCT/US22/71358, which are incorporated herein in their entirely by reference.

### BACKGROUND

### Field

The disclosure relates to methods for providing transcranial magnetic stimulation (TMS) therapy based on electroencephalogram (EEG) data.

### Background

Mental disorders cause serious problems for affected people, their families, and society. Currently, psychiatrists and neurophysiologists treat these disorders with a variety of medications, many of which have significant negative side effects. Mental disorders can be painful, debilitating, and very costly for the affected individual and their family. Approximately one in five adults in the US experiences a mental disorder in a given year. 18.1% of adults in the US experience an anxiety disorder, such as posttraumatic stress disorder, obsessive-compulsive disorder (OCD) and specific phobias. 6.9% of adults in the US have at least one major depressive episode each year. 1.1% of adults in the US live with schizophrenia. In fact, mental disorders are the third most common cause of hospitalization in the US for both youth and adults aged 18-44 and the consequences of lack of treatment are significant. Sadly, suicide is the 10th leading cause of death in the U.S. and the 2nd leading cause of death for those aged 15-24. Each day, approximately 18-22 veterans die by suicide.

A key factor in treatment of mental disorders is proper diagnosis. The standard method of diagnosing mental disorders has been with either the Diagnostic and Statistical Manual of Mental Disorders (DSM) or the International Statistical Classification of Diseases and Related Health Problems (ICD), Chapter 5: Mental and behavioral disorders. Both standards primarily involve diagnosis using conversation with the patient regarding symptoms and behavior. This standard has the disadvantage of being subjectively based on the interviewer's perception, which lessens the diagnostic reliability, sometimes resulting in two clinicians arriving at different diagnoses for the same patient. Additionally, a patient's responses to questions may vary based upon their present situation or life circumstance. Because the DSM and ICD are primarily concerned with the signs and symptoms of mental disorders, rather than the underlying causes, there is a general lack of pathophysiological understanding of mental disorders.

It is apparent that a repeatable and reliable system for the diagnosis of mental disorders that is based on measurable data, independent of the biases and interpretation of an interviewer or a fluctuating situational condition of the patient, would provide significant benefit to patients and to the psychiatric community.

Treatment of these disorders with magnetic fields, such as those generated by transcranial magnetic stimulation (TMS), may generate positive therapeutic responses.

TMS is a generally known non-invasive procedure that uses magnetic pulses to stimulate nerve cells and neuronal circuitry in the brain to improve mental disorders such as depression. Magnetic pulses delivered at regular intervals, described as repetitive magnetic pulses, are referred to as rTMS. Traditionally, coils (e.g., a large electric coil) have been used to generate rTMS. The large electric coil (e.g., electromagnetic coil) may be placed against a patient's scalp when applying stimulation. Some studies have shown that rTMS can reduce the negative symptoms of schizophrenia, obsessive compulsive disorder (OCD), and depression under certain circumstances.

Magnetic fields, especially time and magnitude-varying magnetic fields, can also be generated by movement of one or more magnets. For example, some devices deliver TMS by using rotating permanent magnets where magnets are positioned around a patient's head to deliver TMS towards regions of the patient's brain to stimulate nerve cells in those specific regions of the brain. In some of these examples, rTMS uses an electromagnet placed on the scalp that generates a series of magnetic field pulses roughly the strength of an MRI scan.

There are also normative electroencephalogram (EEG) databases that have been in existence for several years at a few universities. The normative EEG databases are typically accompanied by clinical scores or evaluations of some kind for EEG data that was collected from patients. These normative EEG databases have been used to aid medical professionals in evaluating neurological statuses of patients prior to and after therapy to adjust therapy as needed based on EEG biofeedback. This deeper knowledge can benefit patients, medical professionals (e.g., clinicians), and the greater neurological or neurotherapy field. These databases are usually based on a wide age range from children up to adults. Examples of universities having these normative EEG databases include New York University (NYU) and the University of Maryland.

What is needed are methods that customize application of TMS based on normative EEG data.

### SUMMARY

The disclosure relates generally to methods for providing transcranial magnetic stimulation (TMS) based on normative electrophysiological measurement such as electroencephalogram (EEG) data. The normative database allows the systems and methods to assess deviations from expected normal parameters in a population.

Methods are provided for delivery of TMS based on EEG data. EEG data may be captured from a patient and may be analyzed for EEG characteristics relating to a brain state of the patient. A TMS treatment protocol may be developed or proposed based on the EEG characteristics and the brain state. Synchronized transcranial magnetic stimulation may also be provided based on the determined TMS treatment. In some configurations, the EEG data analysis may be compared against EEG data modelling for the patient. Additionally, the brain state may be reported as a brain health indicator for the patient based on the EEG data. Diagnosis may also be provided for the patient based on the EEG data. EEG data may also be recaptured from the patient and the TMS treatment may be reevaluated and revised based on the recaptured EEG data. Other neuromodulation treatment may also be provided based on the EEG data.

Methods can also deliver or administer TMS based on normative EEG data. The methods use a portable neuro-electroencephalogram synchronization therapy (NEST) system for capturing the EEG data from a patient in communication with a normative EEG database which is operable to customize the application of TMS based on the EEG data compared to the normative EEG data from the database. Under the method, a TMS treatment system can receive EEG data from the portable NEST system. The electrophysiology database and customized TMS treatment system may determine a TMS treatment based on the received EEG data. The portable NEST system may provide synchronized transcranial magnetic stimulation based on the determined TMS treatment.

In some methods, the EEG data may include EEGs from before and after use of the portable NEST system to indicate a set of effects of the TMS treatment. The EEG data may be stored in an object storage database as a set of objects. The portable NEST system may also provide the synchronized transcranial magnetic stimulation relating to a defined treatment protocol. Additionally, the portable NEST system may have a mobile design developed through miniaturization and compacting of components within the portable NEST system allowing for use of the portable NEST system at various locations and when the patient may be in-transit between locations. In some embodiments, the portable NEST system may be configured as a wearable headgear that provides the TMS treatment during mobility of the patient. A portable power source can also be provided. Safety features can also be provided to prevent misuse of the device.

Under the methods, artificial intelligence can also be used for determining a diagnosis and/or determining and recommending the treatment for the patient.

Additionally, the electrophysiology database and customized TMS treatment system may provide a set of aggregated data analytics on the EEG data from a set of multiple patients that may be used as an input for at least one of determining a diagnosis and recommending a treatment for the patient. The electrophysiology database and customized TMS treatment system may generate predictive analytics based on the aggregated data analytics that may be used to identify patients and provide appropriate treatment for the identified patients based on the EEG data. Additionally, the electrophysiology database and customized TMS treatment system may apply big data analytics to model various treatments in terms of predicted impacts to patients and results of treatments.

Methods for recommending TMS based on EEG data are disclosed.

The second system may be used under the methods that includes an electrophysiology database and customized TMS treatment system for receiving the EEG data from the portable NEST system, and the electrophysiology database and customized TMS treatment system may determine the TMS treatment based on the received EEG data.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
U.S. Pat. No. 9,962,555 issued May 8, 2018 to Charles et al.; and
U.S. Pat. No. 10,835,754 issued Nov. 17, 2020 to Charles et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a diagrammatic environment view that depicts an exemplary portable NEST system and an electrophysiology database and customized TMS treatment system communicating via a network, and communicating with various other systems, devices, processes, and information sources according to embodiments of the disclosure;

FIG. 2A is a diagrammatic view that depicts examples of the portable NEST system of FIG. 1 according to embodiments of the disclosure;

FIG. 2B is a diagrammatic environment view that depicts an exemplary portable NEST system and electrophysiology database and customized TMS treatment system communicating via a network, and communicating with various other systems, devices, processes, and information sources according to embodiments of the disclosure;

FIG. 2C is a diagram illustrating a network environment for devices and software;

FIGS. 3A and 3B are example graphs of wave signals or impulses from a conventional rTMS system;

FIGS. 3C and 3D are example graphs of wave signals or impulses from the portable NEST system according to embodiments of the disclosure;

FIG. 4A is an example graph of a frequency spectrum from a conventional rTMS system;

FIG. 4B is another example graph of a frequency spectrum from the portable NEST system according to embodiments of the disclosure;

FIG. 5 is a graph view that depicts a reporting screenshot of a burst histogram of a sample EEG according to embodiments of the disclosure;

FIGS. 6, 7A-B, 8A-B, and 9A-B depict exemplary reporting screenshots for quantitative electroencephalogram (QEEG) relating to magnitude spectra, relative power, and global spectral analysis according to embodiments of the disclosure;

FIG. 10 depicts an exemplary reporting screenshot of pre and post stimulation according to embodiments of the disclosure;

FIGS. 11A-B and 12A-B depict exemplary reporting screenshots for QEEG relating to magnitude spectra and relative power according to embodiments of the disclosure; and

FIG. 13 is a flow chart illustrating a set of operations for an EEG and TMS process according to embodiments of the disclosure.

### DETAILED DESCRIPTION

The disclosure relates to an electroencephalogram (EEG) and transcranial magnetic stimulation (TMS) process. In examples, the EEG and TMS process may be executed by any combination of a neuro-electroencephalogram synchronization therapy (NEST) system, an electrophysiology database and/or a TMS treatment system (optionally using an electrophysiology database and optionally providing for customized, patient-specific TMS treatment protocols, and/or optionally using a combined electrophysiology database and customized TMS treatment system), and/or any other computing device.

The portable NEST system may be easily carried or moved because the device or system may be lighter, compact, transportable, mobile, lightweight and/or smaller than typical or standard devices or systems. The portable NEST systems may include wearable NEST systems, NEST systems configured as headgear, positionable and removable NEST systems (e.g., may be conveniently positioned on or above a chair or seat and then easily removed from the chair or seat), modular NEST systems (e.g., capable of being integrated into another device or system), etc. The portable NEST system may be sized to have a maximum volume or a dimension range, such as about 285 mm length by about 194 mm width by about 188 mm height.

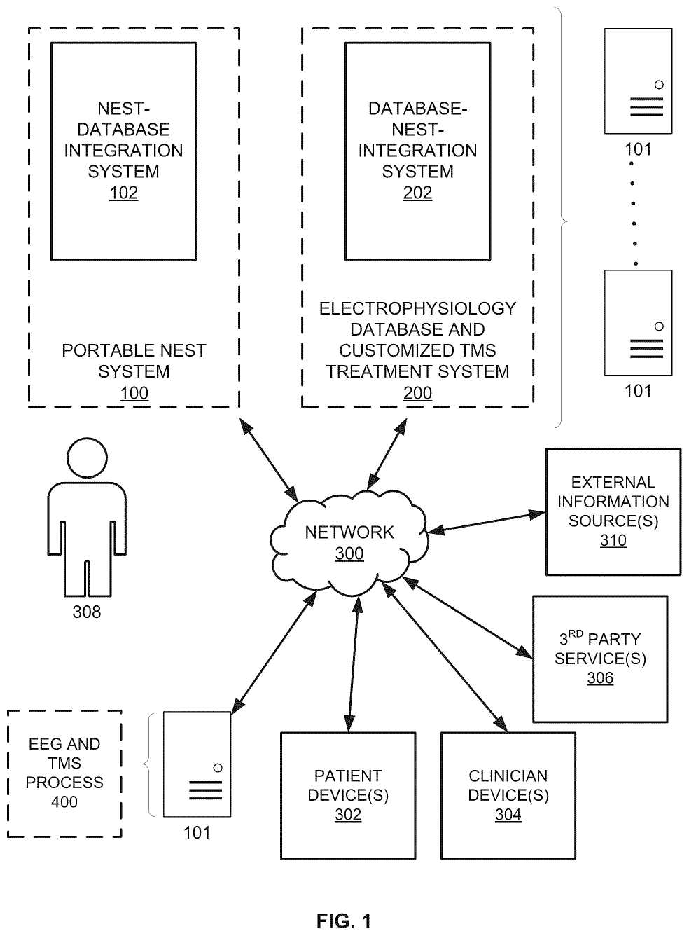

Turning now to FIG. 1 an example environment. The example environment includes a portable NEST system 100 with a NEST database (e.g., NEST-database integration system 102) in communication with a network 300. The network 300 is also in communication with an electrophysiology database and customized TMS treatment system 200 which includes a database integration system (e.g., Database-NEST integration system 202). One or more servers 101 can be provided in communication with the network 300 and/or components in communication with the network 300. Additional components in communication with the network 300 include, for example, external information source(s) 310, third party service(s) 306, clinician device(s) 304, and patient device(s) 302. An EEG and TMS process 400 may run across any combination of the systems and devices. The EEG and TMS process 400 may be run across any combination of an EEG system (such as a portable NEST system 100), an electrophysiology database and customized TMS treatment system 200, and/or another computing device (e.g., one or more servers 101 shown in FIG. 1). The EEG and TMS process 400 may be executed and run on the various systems and computing devices via a network 300. In FIG. 1, the portable NEST system 100 may communicate with the electrophysiology database and customized TMS treatment system 200 according to example embodiments of the disclosure. The portable NEST system 100 may use a NEST-database integration system 102 to communicate and interact with the electrophysiology database and customized TMS treatment system 200 via the network 300. Similarly, the electrophysiology database and customized TMS treatment system 200 may use a database integration system (e.g., Database-NEST integration system 202) to communicate and interact with the portable NEST system 100 via the network 300. Other devices (e.g., various computing devices or mobile devices) such as patient device(s) 302, clinician device(s) 304, and third-party service(s) 306 may communicate with the electrophysiology database and customized TMS treatment system 200 and the portable NEST system 100. The electrophysiology database and customized TMS treatment system 200 and the portable NEST system 100 may also communicate and interact with external information source(s) 310 via the network 300. As shown in FIG. 1, the portable NEST system 100 may be positioned on the head of a patient 308.

The portable NEST system 100 may capture EEG data from a patient 308. The electrophysiology database and customized TMS treatment system 200 may receive the EEG data from the portable NEST system 100. The electrophysiology database and customized TMS treatment system 200 may determine a TMS treatment based on the received EEG data. The portable NEST system 100 may provide synchronized transcranial magnetic stimulation (sTMS) based on the determined TMS treatment. The electrophysiology database and customized TMS treatment system 200 may also access the EEG data for a patient 308 and may classify the patient 308 as having a particular brain type based on the EEG data. The electrophysiology database and customized TMS treatment system 200 may determine a TMS treatment based on the classified brain type and the EEG data. The electrophysiology database and customized TMS treatment system 200 may recommend sTMS based on the determined TMS treatment. In some examples, the portable NEST system 100 may capture the EEG data from the patient 308 that may be accessed by the electrophysiology database and customized TMS treatment system 200. The portable NEST system 100 may provide the recommended synchronized transcranial magnetic stimulation to the patient 308. The electrophysiology database and customized TMS treatment system 200 may access the EEG data for a patient 308 and may analyze the EEG data to indicate brain health of the patient 308. The electrophysiology database and customized TMS treatment system 200 may determine TMS treatment based on the brain health indication and the EEG data. The electrophysiology database and customized TMS treatment system 200 may recommend sTMS based on the determined TMS treatment. In some examples, the electrophysiology database and customized TMS treatment system 200 may provide reporting of the brain health indication in the form of a brain health indicator for the patient 308. The electrophysiology database and customized TMS treatment system 200 may also use the brain health indication for diagnosing the patient 308. The electrophysiology database and customized TMS treatment system 200 may access the EEG data for a patient 308 and may provide bursting analysis of the EEG data. The electrophysiology database and customized TMS treatment system 200 may determine a TMS treatment based on the bursting analysis and the EEG data. The electrophysiology database and customized TMS treatment system 200 may also recommend sTMS based on the determined TMS treatment. Bursting analysis may be used for diagnosing the patient 308. The bursting analysis may be used for determining treatment parameters. The portable NEST system 100 may also capture EEG data from a patient and have one or more rotating magnets for generating an alternating magnetic field. The portable NEST system 100 may determine a TMS treatment recommendation based on the EEG data or may receive the TMS treatment recommendation from a second system based on the EEG data. The portable NEST system 100 may provide sTMS by using the rotating magnets to generate the alternating magnetic field based on the determined TMS treatment or the recommended TMS treatment. The second system may be the electrophysiology database and customized TMS treatment system 200 for receiving the EEG data from the portable NEST system. The electrophysiology database and customized TMS treatment system 200 may determine the TMS treatment based on the received EEG data.

NEST System Components

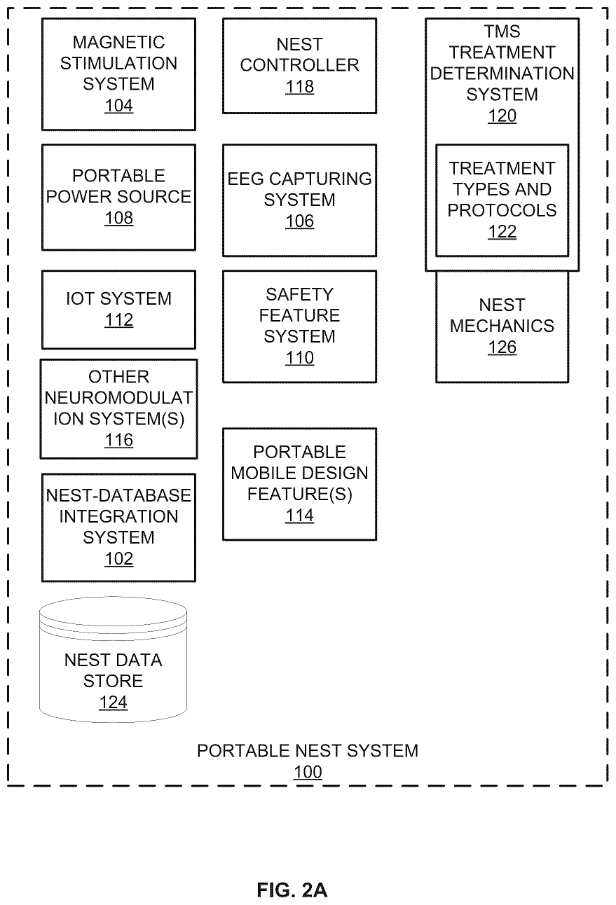

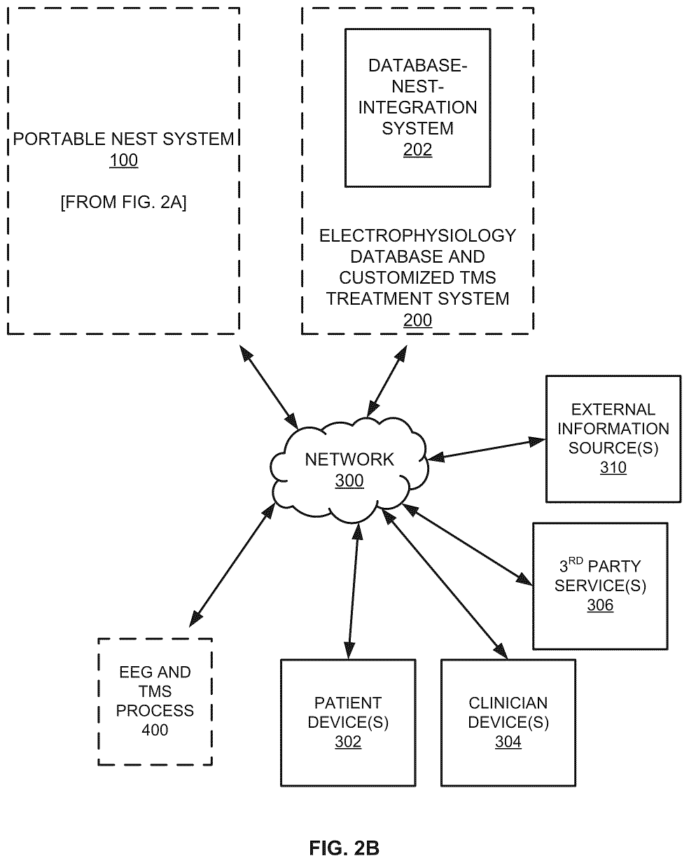

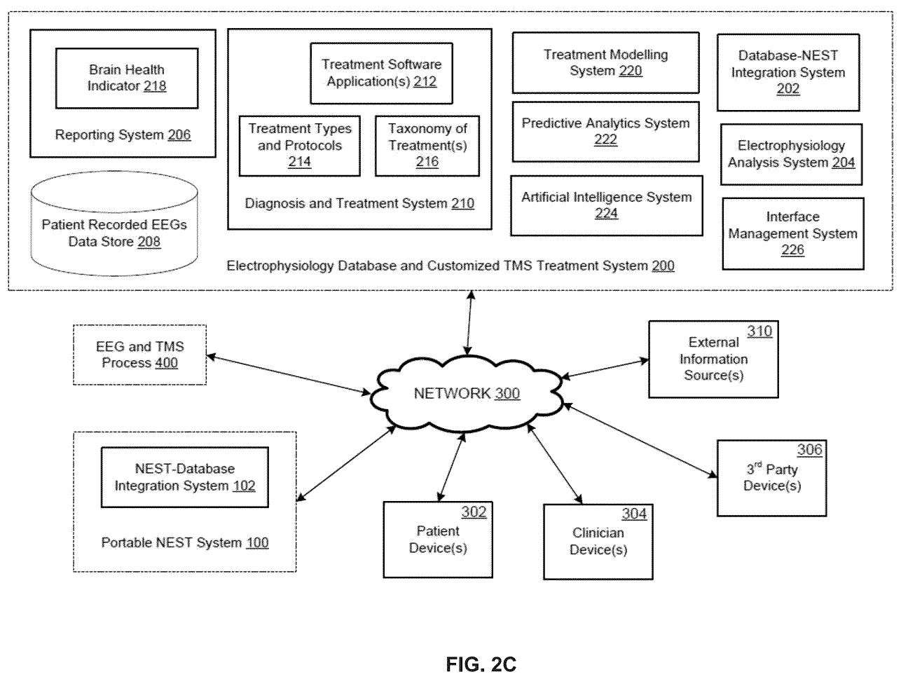

FIG. 2 shows a detailed view of the portable NEST system 100. The portable NEST system 100 may include the NEST-Database integration system 102 (e.g., portable NEST system software that enables integration with software of the electrophysiology database and customized TMS treatment system 200), a magnetic stimulation system 104 (e.g., including one or more magnets that may provide sTMS), an EEG capturing system 106 (e.g., detecting and capturing EEG data from the patient 308 using sensors such as electrodes placed along scalp of the patient 308), a portable power source 108 (e.g., portable-type power source such as a battery), a safety feature system 110 (e.g., one or more safety mechanisms that may prevent or avoid misuse of the portable NEST system 100 such as a locking mechanism), an Internet of Things (IoT) system 112 (e.g., including various components that may provide IoT capabilities such as relating to connecting the portable NEST system 100 to the network 300), portable/mobile design feature(s) 114 (e.g., one or more features that enable for portability and mobility use of the portable NEST system 100 such as miniaturizing of some features or components), other neuromodulation system(s) 116 (e.g., systems that provide other therapies such as light stimulation therapies), a NEST controller 118 (e.g., controller may be used for managing and directing one or more components of the portable NEST system 100), a TMS treatment determination system 120 (e.g., system for determining repetitive TMS (rTMS) and sTMS that may be based on the EEG data obtained by the EEG capturing system 106) having treatment types and protocols 122 (e.g., information and/or data related to treatment types and protocols as described in the disclosure that may be used by the TMS treatment determination system 120), a NEST data store 124 (e.g., storing any and all data related to use of the portable NEST system 100 and possibly some data received from other systems and/or devices such as received from the electrophysiology database and customized TMS treatment system 200), and NEST mechanics 126 (e.g., various mechanical parts relevant to configuration of the portable NEST device, such as an example portable headset device, for use by the patient 308). The network 300 can be connected to one or more patient device(s) 302, one or more clinician device(s) 304, third party service(s) 306, and external information source(s) 310. The headset device provides a support framework with attached electronic devices that is worn on the head of the user. The form factor of the headset can be a cap, helmet, or hat.

Comparison of Wave Signal Plots Against Typical rTMS Plots

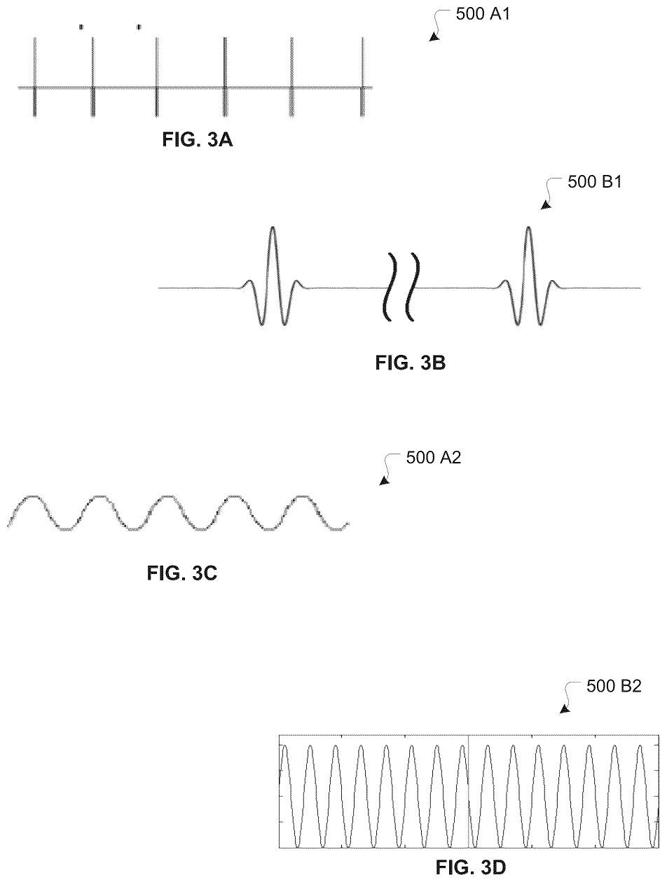

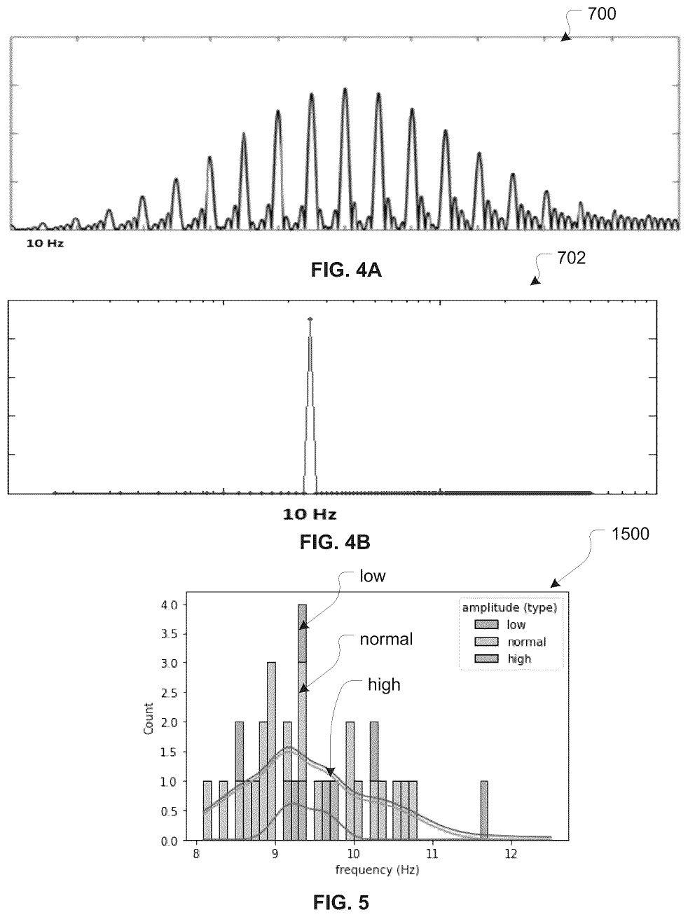

There are several advantages to using the portable NEST system 100 compared to the conventional rTMS devices. Referring now to example implementations, FIGS. 3C, 3D, and FIG. 4B show pulse waveform/shapes and a frequency spectrum, respectively, of the portable NEST system 100 according to one or more embodiments of the disclosure. The pulse waveform/shapes and the frequency spectrum of the portable NEST system 100 may be compared to waveform/shapes and a frequency spectrum for a conventional rTMS system. Specifically, in FIGS. 3A and 3B, there are example graphs of the pulse waveforms/shapes for a conventional rTMS system. In FIGS. 3C and 3D, there are example graphs of the pulse waveforms/shapes for the portable NEST system 100 according to one or more embodiments of the disclosure. The conventional pulsed rTMS waveforms/shapes may be compared to the NEST pulse waveforms/shapes of the portable NEST system 100. Specifically, the conventional rTMS shown in FIGS. 3A-3B may tend to produce pulses 500A1, 500B1 having sharp impulses (e.g., sinusoidal shape with a period of about 300 microseconds). In contrast, the portable NEST system 100 may produce pulses 500A2, 500B1 as shown in FIGS. 3C-3D that may be purely sinusoidal waves. FIG. 4A shows an example frequency spectrum 700 graph from the conventional rTMS system in which the power of conventional rTMS is spread across all harmonics of the pulse frequency. FIG. 4B shows a frequency spectrum 702 for the disclosed portable NEST system which shows that all the power is concentrated at a single frequency. As shown in the frequency spectrum of FIG. 4A, the conventional rTMS may frequently produce a frequency spectrum 700 with power of rTMS spread across most (if not all) harmonics of pulse frequency whereas in FIG. 4B, the portable NEST system 100 may produce a frequency spectrum 702 with most (if not all) of the power of the portable NEST system 100 being concentrated at a single frequency. In this example, the conventional rTMS and the portable NEST system 100 may produce pulses at about ten Hertz.

Medical Device for Monitoring Treatments

The portable NEST system 100 may also be used for monitoring the effects of treatments. The portable NEST system 100 may be used to monitor the effects of treatments that are still in a trial stage such as drugs, other treatment devices, and protocols on brain states of patients (e.g., during U.S. Food and Drug Administration (FDA) clinical trial stages for drugs, devices, and/or protocols). Approval of treatments by government agencies may be based on the trial requirements for each government agency (e.g., FDA regulations for approving trials based on data). The portable NEST system 100 may be useful in providing compliance testing, safety testing, efficacy testing, biocompatibility testing, etc. required for approval of different types of treatments (e.g., drugs, devices, and/or protocols) during a clinical trial. As will be appreciated by those skilled in the art, different patient populations may have different requirements relating to approval by the government agency (e.g., FDA regulation approval). More specifically, patients with PTSD may have different compared patients with ADHS. Additionally differences can be based on age, gender, and other factors.

Treatment of Disorders

The portable NEST system 100 may provide a synchronous TMS treatment that may be used to treat several disorders. These disorders may include ADHD, PTSD, traumatic brain injury, etc.

Improve and Maintain Brain Performance

The portable NEST system 100 may also include a portable unit operable to deliver sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the portable unit device may provide the sTMS to improve or maintain brain performance. The portable NEST system 100 may improve performance for patients. For example, performance may be improved with regard to focus, concentration, general wellness, physical performance, academic performance, sports, athletic performance, etc. These improvements may occur in the short term and long term after using the portable NEST system 100. In addition, these improvements may be cumulative. The EEG and TMS process 400 may use the portable NEST system 100 for optimizing brains of patients who may want to improve performance. The portable NEST system 100 has resulting effects that may be provided such as causing brain activity to be more rhythmic which improves performance in several categories (e.g., improved sports and academic improvement). The portable NEST system 100 may also be used in a variety of ways such as using both the portable NEST system 100 and conventional rTMS to provide improved brain performance. Use of the rTMS may be a non-invasive brain stimulation where a changing magnetic field may be used to cause electric current at a specific area of the brain through electromagnetic induction. In some examples, where subjects or users may be in adverse circumstances (e.g., space travel, otherworldly habitation, or life in stressful environments, such as for deployed soldiers or on military bases), these users or subjects may use the portable NEST system 100 to provide sTMS that may relate to treatment types and protocols such that the portable unit device may provide the sTMS to improve or maintain brain performance of the users or subjects.

Classification of Brain Personality and Characteristics (Emitter vs. Absorber)

The portable NEST system 100 may also have a portable unit device operable to deliver sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the portable unit device may provide the sTMS as a treatment based on a brain classification. In general, the brain may affect personality in terms of strengths and weaknesses. For example, brains may be classified into two different categories: highly rhythmic brain (low energy brains) and high energy brains (chaotic brains that may be less rhythmic brains). The highly rhythmic brain may refer to an absorber-type of personality and the high-energy brain may refer to an emitter-type of personality. Absorber patients and emitter patients may have different personality traits relating to being either the absorber or the emitter.

The absorber personality may be associated with an extremely rhythmic brain, which may tend to be a lower energy brain that may mean that the brain may be relatively more efficient. The EEG and TMS process 400 (e.g., using the treatment types and protocols 122, 214) may be used to determine a personality of a patient who has a rhythmic brain (e.g., identify patients as having absorber personality based on EEG data). The absorber personality may have traits that may include being calmer, may tend to be less showing of emotions (e.g., less expressive of emotions), and may tend to get much less excited about certain things. For example, an absorber person may not be as happy or sad and may tend to be a lot more even-keel, middle of the road. The absorber personality may tend to prefer to focus on one individual task at the exclusion of all other tasks (e.g., focus exclusively on studying for one exam at the exclusion of other projects). In local school systems, the absorber personality may tend to prefer other absorbers who may be rule followers, soldier-type personalities, follow the recipe type personalities, etc.

The emitter personality may relate to a high energy EEG. The emitter personality may have traits that may tend to be relatively expressive in their emotions. For example, if an emitter personality is having a bad day, one may easily notice from the expression on their face. The emitter personality may tend to be more creative and may be more out-of-the-box thinker types. While the absorber personality may be conflict-avoidant, the emitter personality may enter into conflict if they feel a need to make a point. The absorber personality may work on single tasks whereas the emitter personality may prefer to move between multiple tasks.

The EEG and TMS process 400 may be used to find correlations between professions or various roles, these brain types, and/or other identified endophenotypes. Typically, when referring to people with different brain types, there may be some correlation that most (if not all) engineers may be absorbers whereas executives (e.g., CEO) may be typically found to be emitters. This may follow along as well with athletes and soldiers. For example, from EEG recordings of a number of professional football players, it may be found that in most (if not all) cases, it may be determined that the football players are absorbers. There is also some percentage correlation between the football players and some brain injury. A majority of soldiers may be found to have a relatively high or highly rhythmic brain (especially high performer soldiers). The highly rhythmic brain may be found in EEGs of special operators in armed forces and other professionals who were considered to be at the top of their field.

The EEG and TMS process 400 and specifically the portable NEST system 100 may be used to improve performance. The improved performance may result from making the brain more rhythmic. This may also be described as making a patient more of an absorber. These changes to the brain in terms of being more rhythmic may not have a dramatic personality effect but the patient may become slightly better at concentrating and focusing on single tasks and the patient may be able to improve performance of actions that may require focus better than before which may help with academics and athletics. In some examples, it may not be necessary to distinguish much between the treatments of brains for improving athletic performance versus treatment of brains for improving academic performance. The treatment goals may be similar in trying to make brains more rhythmic in nature, which may improve both academic and athletic performance.

There may be various examples for classifying brain characteristics. In some examples, classification of brain characteristics may include or may be based on frontal generation classification, low voltage, left or right posterior alpha generation, and/or personality characteristics (e.g., personality characteristics may be identified via surveys collected concurrently with EEG monitoring such that the EEGs may be captured by the portable NEST system 100). Classification of brain characteristics may be based on identified disruptions of function (e.g., synchronous frontal theta due to head injury may provide a different personality classification due to disruptions of normal cognitive and sensory processes).

Optimal Brain Profile

The EEG and TMS process 400 may be used with the portable NEST system 100 and the electrophysiology database and customized TMS treatment system 200 to treat a patient's brain towards an optimal brain profile. There is a balance where too rhythmic brain activity or excessive frontal rhythmicity may result in a tendency towards depression. For example, some patients may be candidates for low mood (e.g., some type of depression). The EEG and TMS process 400 may include activation of magnets (of the magnetic stimulation system 104) in different ways instead of simply treating at an intrinsic frequency. The EEG and TMS process 400 may provide treatment at random frequencies or may treat off frequency. Magnetic stimulation treatment may be analogized as pushing a swing in a way that may not be meant to be pushed. For example, applying magnetic stimulation that may not match the intrinsic frequency may actually disturb and “kick” the brain slightly to get the brain towards an ideal frequency (e.g., based on optimal brain profile).

With regard to coherence (brain activity oftentimes), the EEG and TMS process 400 may find chaotic activity. The chaotic activity may not be throughout the entire brain but may be only in particular regions of the brain. The EEG and TMS process 400 may use the portable NEST system 100 (e.g., specifically the TMS treatment determination system 120 with the magnetic stimulation system 104) to target those chaotic regions to improve communication coherence of the brain.

The EEG and TMS process 400 may also be used with autistic patients that may have dramatically chaotic EEG activity. Autistic patients may typically have high-energy brains (e.g., hyper-connectivity posteriorly and poor anterio-posterior connectivity). The high-energy brains may be associated with behavioral problems of autistic patients because they are trying to deal with their brain activity being so chaotic. For example, some autistic patients tend to do stimming, which may be tending to rhythmic activity. 2304 the autistic patients (e.g., autistic children) may rock back and forth in a rhythmic way and/or may tap their fingers in a rhythmic way such that they may do anything they can in a rhythmic way. The autistic children may be attempting to, not consciously, become more rhythmic by performing these actions (e.g., rocking or through other motions). The stimulation applied by the portable NEST system 100 may improve disrupted cortical connectivity and reinforce synchronous rhythms in autistic patients. The autistic patients may be comforted by the pulse of the stimulation (e.g., whether from a large coil or magnets on a patient's forehead). The resultant changes in network connectivity and function may provide more coherent sensory and cortical communication, resulting in improvements in cognition and processing as well as resultant behavioral improvements.

Environments and Variety of Uses

The portable NEST system 100 may be used in various possible ways in applying TMS (e.g., rTMS and/or sTMS). The portable NEST system 100 (e.g., as a portable unit device) may be integrated and used in various user environments. The portable NEST system 100 (e.g., battery powered NEST device) may be configured to be used in different environments such as at home, while watching television, while meditating, in bed, in a car, etc. The portable NEST system 100 may also provide stimulation to the brain in order to make it produce the optimal variety of brainwaves for aiding performance in whatever activity a patient may likely be performing. In some configurations, the portable NEST system 100 improves relaxation on airplanes, focus while driving, or any number of mental states for a specified task or activity. Over the course of the 24-hour day, the patient's brain may keep various brainwaves active (e.g., typically within five bands—within delta, theta, alpha, beta, gamma bands). Delta and theta activity may be most generally present during shallow and deep stages of sleep, depending on sleep stage. Alpha, beta, and gamma activities may be most generally dominant in the EEG during wakefulness. Alpha activity may be generally present during states of reflection and during eyes closed periods. Beta activity may be present during waking, active states. Gamma activity may be generally present during task learning and acquisition, and in contrast to other EEG activities generally only appears in local tissue and disperse between cell layers. Depending on what the patient may be doing, some brainwaves may be more active in certain areas of the brain while other brain waves may be less active in other areas of the brain, while activity within these brainwave bands may not be fully “switched off” per se. In specific examples, the portable NEST system 100 may be used in first-class airlines seats of jets, used while sleeping at night, and/or used while undergoing a test. The portable NEST system 100 may be used while performing an activity requiring focus and/or concentration such as used with a race car driver, a truck driver, etc. The portable NEST system 100 may also be used in virtual reality environments (e.g., utilizing virtual reality technologies).

Patients may use the portable NEST system 100 in combination with other modalities. The portable NEST system 100 may also be used along with other stimulation that might augment entrainment effects, such as through rhythmic stimulation as sound, lights, other stimulations, and/or other therapies.

The portable NEST system may be used with virtual reality (e.g., used with a virtual reality device such as virtual reality goggles). There may be various activities that patients may engage in while undergoing stimulation using virtual reality. The portable NEST system 100 may be a separate device, which is worn in conjunction with or integrated with the virtual reality device. The portable NEST system 100 may incorporate virtual reality capabilities such that the patient 308 may only need to operate a single device to obtain the benefits of both virtual reality and the NEST therapy.

The portable NEST system 100 may be used in different situations or different scenarios. The portable NEST system 100 may be used at a storefront at the mall (e.g., people may go in for a regular back massage and they may obtain a brain massage with the portable NEST system 100). For example, a person may feel that they are having trouble concentrating or may not feel well and the person may stop at a store for twenty minutes of NEST treatment in order to improve their mental health. The EEG and TMS process 400 may include use of the portable NEST system 100 combined with a massage or may be combined with other relaxation treatments (e.g., sensory deprivation chamber). The portable NEST system 100 may be used at home, during sports events, and similar activities.

The portable NEST system 100 may be used with patients when they sleep or as they fall asleep. Use of the portable NEST system 100 may improve sleep. For example, brains of absorbers (e.g., rhythmic brains) may tend to have trouble sleeping (e.g., have insomnia) whereas high-energy brains (e.g., emitter-type of personality) may have an easier time sleeping (e.g., greater ease in falling asleep). By shifting a patient's brain to a less rhythmic state or higher energy state, the treatment by the portable NEST system 100 may help with focus and may help with sleep.

The EEG and TMS process 400 and specifically the portable NEST system 100 may be used to help patients fall asleep, achieve deeper sleep states, and treat patients when they are sleeping. For some people, there may be feedback from running stimulations such that they may find that the TMS treatment may disrupt their sleep because the stimulation may accentuate their normal resting awake brain waves as opposed to frequencies for sleep. However, the feedback with sleep may be resolved by varying frequency of the rotating magnets of the magnetic stimulation system 104 such that instead of accentuating intrinsic alpha frequency, the magnetic stimulation system 104 may reinforce delta and theta bands which may be typically observed during stages 2 and 3 of sleep. Additionally, stimulation may move into upper alpha and lower beta frequency ranges during REM sleep, to facilitate or augment resultant dreaming. The stimulation frequency may be modeled to follow the individual's sleep cycle and EEG rhythms specific to the patient corresponding to each stage of sleep. The EEG and TMS process 400 may use the portable NEST system 100 to record EEGs (e.g., using the EEG capturing system 106) during sleep and then may gradually adjust magnetic fields to accentuate various frequencies (e.g., reinforce delta and theta bands during stages 2 and 3 of sleep and move into upper alpha and lower beta frequency ranges during REM sleep) that may be used to bring patient to, for example, rapid eye movement (REM) sleep or deep sleep. The EEG system may indicate when the subject is transitioning between states, and then stimulation may be used to encourage observed transition. As stimulation is subthreshold, patients may not feel the magnetic field (e.g., do not feel electric currents generated) from the portable NEST system 100. As described in the disclosure, electric currents may be relatively small with the portable NEST system 100. In contrast, conventional rTMS devices with a coil may sit above the forehead and may apply stimulation that may not be conducive to sleep.

Provide Neuromodulation with Stimulating Forces (e.g., Magnetic Stimulating Forces)

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols 214 of the electrophysiology database and customized TMS treatment system 200 described in the disclosure) such that the sTMS may include magnetic stimulating forces that may provide neuromodulation. The portable NEST system 100 may use the magnetic stimulation system 104 to provide neuromodulation with stimulation forces (e.g., magnetic stimulation forces). The portable NEST system 100 may use the magnetic stimulation system 104 to apply a magnetic force in cortical tissue and then may change or influence how that cortical tissue may behave. The portable NEST system 100 may apply one force as magnetic stimulation that may be generated at a specific frequency. The portable NEST system 100 may apply overlapping forces. The portable NEST system 100 can also apply multiple overlapping frequencies, or different frequencies from each rotating magnet. For this example, the portable NEST system 100 may apply the same multiple magnetic pulses in different locations or a combination of forces. The portable NEST system 100 may use the same focal magnetic pulses in different locations or a combination of forces.

With magnetic stimulation, the EEG and TMS process 400 may also calculate the intrinsic frequency (e.g., using algorithms). The EEG and TMS process 400 may identify a frequency where the energy of the EEG may be at its maximum. This may be how an optimal frequency setting may be determined for each patient. When referring to applying overlapping forces, the magnetic stimulation system 104 of the portable NEST system 100 may use any number of magnets such as three or more magnets to apply the overlapping forces. The magnets themselves may each have their own alternating magnetic field. When the magnets are rotating near each other, the fields of the magnetic field vectors of two magnets may be added together. Thus, there may be constructive and destructive interference. Positions of magnets, magnet types, magnetic frequencies, or the phase between the magnetic fields between two magnets may be changed in order to accentuate and to stimulate some areas of the patient's brain more than other areas of the brain.

The magnetic stimulation system 104 may use various types of magnets and arrangements of magnets. The magnets of the magnetic stimulation system 104 may be arranged as or positioned in a Halbach array (e.g., arrangement of permanent magnets that may augment a magnetic field on one side of the array while cancelling field to near zero on other side of the array). The magnets may be changed around to where the magnets may accentuate the magnetic field in one direction and reduce it in another. Rotating magnets may be used along with the coil (e.g., large coil) to generate a magnetic field that has unique characteristics relating to overlapping forces approach.

The portable NEST system 100 may be used at a fixed location that may be considered generally ideal for most patients. The portable NEST system 100 may be configured to change the position of the magnetic stimulation system 104 (e.g., specifically the magnets) to treat specific regions of the patient 308 that may be based on each patient and/or a mental disorder. In general. The EEG and TMS process may be focused on the patient's brain in making treatment determinations (e.g., using the TMS treatment determination system 120). The TMS treatment determination system 120 may monitor and use neuronal activity for determination and directing the location of treatment and the treatment frequency based on the brain itself as part of the EEG and TMS process 400. Particular indications may also be used in this assessment and determination. Additionally, the magnets may be positioned to target a specific region of the patient's brain.

For example, a traumatic brain injury may be an example of a strategic location of treatment (e.g., relocation and/or movement of magnets for directing stimulation). This may be due to an actual injury to the brain itself such that stimulation of the area around the brain injury may help with the restoration of disrupted connectivity of neurons because some neurons and their networks are damaged, killed or destroyed from that trauma. The TMS treatment determination system 120 may be used to determine and direct the magnetic stimulation system 104 (e.g., magnets) to be positioned to provide stimulation to the particular damaged area (e.g., magnetic field to damaged area). Fixed ideal locations (e.g., one location or two locations such as prefrontal cortex, dorsal lateral prefrontal cortex, motor cortex, and the like as described in the disclosure) may be treated where the magnets may be fixed in positions from patient to patient.

The EEG and TMS process 400 may include recording of patient's EEG and then the EEG and TMS process 400 may indicate information about the patient based on EEG (e.g., analysis of EEG data of the patient 308). The EEG and TMS process 400 may set the frequency or phase (e.g., using the TMS treatment determination system 120).

Use with Other Neuromodulation Technologies

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the sTMS may include magnetic stimulating forces that may provide neuromodulation that may be combined with one or more therapies. Other neuromodulation technologies may be used with the portable NEST system 100. These other neuromodulation technologies may include electrical (e.g., transcranial direct current stimulation), light stimulation therapies (e.g., photons), vibration (e.g., massage), and/or similar stimulating force that may stimulate cortical tissue. The portable NEST system 100 may be configured and/or other devices may be provided in combination with the portable NEST system 100 allowing for these other neuromodulation technologies (e.g., electrical, light) to be applied with the magnetic stimulation.

As described herein other neuromodulation technologies may include virtual reality technology. For example, a patient may view a video that may contain frequencies. A light in the view or a separate light may be set to flash at the same frequency or a harmonic of the patient's brain frequency. The portable NEST system 100 may also be redesigned to incorporate these other mechanisms such as by having headphones, goggles, vibration mechanisms, or the like that may allow for the portable NEST system 100 to impart these other neuromodulation technologies or mechanisms.

In general, the EEG and TMS process 400 may utilize these other mechanisms or therapies that may relate generally to locomotive activity (e.g., rhythmic activity) because rhythmic activity may affect the patient's brain. The EEG and TMS process 400 may be used to direct a person to perform an activity that may be a harmonic of the brain in terms of frequency such that the brain's intrinsic frequency may be accentuated. The EEG and TMS process 400 may also utilize software to provide a recommendation and/or direct a patient to run at a running pace equal to the harmonic of the patient's EEG. Running at this harmonic pace may accentuate the rhythmic nature of the patient's EEG more so than if the patient ran at some other random pace or frequency. The portable NEST system 100 may include a magnetic field generator with a system that may allow for performance and/or direction of a rhythmic activity at a specific frequency related to the frequency of the patient's brain (e.g., based on EEG data) such that the patient may be in sync with the magnetic field generated and the rhythmic activity.

Provide Personalized TMS Based on a Determined Frequency

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the provided sTMS may be personalized TMS based on a determined frequency for each patient. The portable NEST system 100 may use the TMS treatment determination system 120 for providing personalized TMS treatment (e.g., rTMS and/or sTMS stimulation treatment) based on a determined frequency. As described in the disclosure, the TMS treatment determination system 120 may be directed to where frequency of magnetic pulses may be determined based on brain resonant phenomenon (e.g., patient intrinsic alpha frequency). The TMS treatment determination system 120 may target location along with that or other potential technologies for treating the patient's brain. In some examples, the TMS treatment determination system 120 may be used to find an intrinsic frequency in an alpha range, which may be a dominant frequency. The TMS treatment determination system 120 may use a correlation technique that may take essentially small wavelets that may have frequency content at different frequencies and may perform a correlation across the EEG data. The TMS treatment determination system 120 may also utilize various other ways (e.g., curve fitting technique) for determining the dominant frequency or intrinsic frequency.

The magnetic stimulation system 104 may be a system utilizing rotating magnets to stimulate the brain for a number of potential purposes, including treatment of various mental disorders. The magnetic stimulation system 104 may rotate permanent magnets that may not be directed at a patient's intrinsic brain frequency but instead may generate an arbitrary frequency.

An intrinsic frequency may be difficult to detect for patients and some patients may not have a findable intrinsic frequency. Some autistic patients may not evince the intrinsic frequency because their brain activity may be extremely chaotic or the network underdeveloped. With autism and other examples, an intrinsic frequency and an alpha frequency may not be discoverable. In some examples, older patients may have brain activity that may be at a point where it may be difficult to detect or discover any peak in the alpha frequency (e.g., alpha rhythms). One reason for this is because the dominant frequency may tend to decrease with increasing age and individuals with dementia and Alzheimer's often may have dominant frequencies significantly less than about 8 Hz, entering the theta EEG band. For these examples, the EEG and TMS process 400 may set the portable NEST system (specifically the magnetic stimulation system) to a particular frequency (e.g., also referred to as an arbitrary frequency). This arbitrary frequency may be determined (e.g., by the TMS treatment determination system 120 and the electrophysiology database and customized TMS treatment system 200) as being most ideal for all patients generally and/or most ideal for similar patients based on demographics (e.g., age, gender, ethnicity, etc.) and/or disorders (e.g., depression, PTSD, autism).

Frequency may also be determined using harmonic relationships (e.g., based on heart rate). The EEG and TMS process 400 may record an electrocardiogram (ECG or EKG) (e.g., by using the portable NEST system 100) for determining frequency to use for brain treatment (e.g., may be based on an intrinsic alpha frequency determination). The harmonic relationship may be between a patient's heart rate and their intrinsic alpha frequency. The patient's heart rate may be about one-eighth of the patient's intrinsic alpha frequency such that the magnetic stimulation system 104 may set the frequency of magnet(s) to be the harmonic of the heart rate in the alpha band. A normal intrinsic alpha frequency may be about 9.6 Hertz whereas a normal heart rate may be around 1.2 Hertz. The EEG and TMS process 400 may set the magnet frequency of the magnetic stimulation system 104 to be a harmonic of the heart rate in an alpha band. The EEG and TMS process 400 (e.g., using the TMS treatment determination system 120 with or without the electrophysiology database and customized TMS treatment system 200) to determine and select a frequency near the middle of the alpha band that may be a harmonic rhythm with the patient's heart rate (e.g., based on relationships between heart rate and intrinsic alpha frequency).

The EEG and TMS process 400 may use other body functions as harmonic relationships for determining treatment frequency. For example, heart rate may be about one-eighth of a patient's intrinsic alpha frequency. Breathing rate may be about one-fifth of heart rate. Gastrointestinal movement rate (e.g., relating to movement of food through the body for digestion) may be about one-fifth of breathing rate. Elements in the body may be harmonically regulated because harmonic activity may be lower energy than asynchronous activity. Accordingly, if detection and/or discovery of an alpha frequency cannot be accomplished or may be at least difficult, the EEG and TMS process 400 may use the portable NEST system 100 to measure and obtain other rates and rhythms of the body (e.g., heart rate, breathing rate, etc. as described in the disclosure) to be used in determining the alpha frequency (e.g., based on relationship of alpha frequency with rates and rhythms of the body). The portable NEST system 100 may capture heart rate data (e.g., EKG data) which may be used to determine and set treatment stimulation to a harmonic of the heart rate in the alpha band (e.g., the patient heart rate being about one-eighth of the harmonic).

EEG Used in Determining Treatment

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the provided sTMS may be part of a treatment determined based on one or more electroencephalograms (EEGs) for each patient. The TMS treatment determination system 120 may use EEG data from patients in determining treatment. This may be based on EEG data obtained by the EEG capturing system 106 (e.g., using electrodes). The TMS treatment determination system 120 may also communicate with the electrophysiology database and customized TMS treatment system 200 as part of the EEG and TMS process 400 in determining treatment based on the EEG data. EEG data may be a biometric measurement that may be a measurement of brain function and may improve over time. The portable NEST system 100 may capture input of EEG biometric measurements (e.g., using the EEG capturing system 106), patient's responses to stimulation, patient's performance in an activity, and patient's general behavior that may be used by the TMS treatment determination system 120 in combination with the electrophysiology database and customized TMS treatment system 200 for determining brain treatment. The TMS treatment determination system 120 may allow for use of EEG data from patients to be used for determining frequency that TMS may be set to and may be used to improve mental health of patients. The EEG and TMS process 400 may use the portable NEST system 100 to provide neural EEG stimulation therapy (NEEGST). The TMS treatment determination system 120 may use patient's EEG data to determine an exact frequency to rotate magnets in order to generate synchronized personalized TMS therapy (e.g., low-level therapy). An algorithm may be used for determining frequency of rotation for magnets based on EEG data. This determination may include the TMS treatment determination system 120 and/or electrophysiology database and customized TMS treatment system 200 looking or monitoring for the intrinsic frequency in the alpha range (e.g., may be a dominant frequency). For example, if a patient sits with their eyes closed, relaxed, with an EEG capturing system 106 (e.g., EEG headset) on, the alpha frequency may be determined from the EEG data obtained. In some examples, the alpha frequency may be between eight and thirteen Hertz.

The TMS treatment determination system 120 and/or the electrophysiology database and customized TMS treatment system 200 may identify bursting, high level, rhythmic activity in the EEG data. This bursting may be referred to as the brain's central clock. The EEG and TMS process 400 may have a general goal of finding this bursting in order to determine the alpha frequency. The EEG and TMS process 400 may use different methods that may be used to find the bursting dominant frequency such as by using the fast Fourier transform (FFT) (e.g., an algorithm that may be used to compute discrete Fourier transform (DFT) for sequence or the inverse DFT (IDFT)) to find the dominant frequency in the FFT which may be used as the intrinsic frequency. The EEG and TMS process 400 may use signal processing for finding the dominant frequency. The EEG and TMS process 400 may use a correlation technique (e.g., using the TMS treatment determination system 120 and/or the electrophysiology database and customized TMS treatment system 200). The correlation technique may include taking essentially small wavelets that may have frequency content at different frequencies and performing a correlation across the EEG data. The EEG and TMS process 400 may look for and identify areas where the correlation may be high. If a sufficient number of these highly correlated areas may be found, the frequency at these areas may be obtained and used as the dominant frequency or intrinsic frequency. The EEG and TMS process 400 may utilize various other ways for determining the dominant frequency or intrinsic frequency. A curve fitting technique may be used by the EEG and TMS process 400 for determining the intrinsic frequency or the dominant frequency. These approaches are important and useful for the EEG and TMS process 400 as determining the intrinsic frequency may be difficult where some patients may have relatively low voltage EEGs which can make it difficult to identify the intrinsic frequency (e.g., finding a little burst of alpha frequency may be difficult). The EEG and TMS process 400 may also determine intrinsic frequency by using stimulation to find a ringing effect. Similar to a swing, if a patient is stimulated at a correct frequency, there may be a resulting ringing effect. With the swing, the correct frequency may cause the swing to get higher and higher and it may take a little while for the swing to drop back down. Similarly, by stimulating at the correct frequency, the TMS treatment determination system 120 and/or the electrophysiology database and customized TMS treatment system 200 may discover the ringing effect in the EEG data. After the stimulation ends, the EEG may be relatively rhythmic but then may become less rhythmic after a period of time. The ringing effect may be more pronounced for longer periods of time when running at the correct frequency. In summary, there may be various ways of identifying the correct frequency and/or dominant frequency that may be used for determining the alpha frequency or intrinsic frequency.

A stimulation parameter calculation may be a result of analysis of not only single, but also multiple EEGs. The stimulation frequency may be related to a change of activities in serial EEGs such as in the example where stimulation may be delivered between EEGs and an algorithm may track EEG parameter response to stimulation. Other example uses may not necessarily relate to treatment delivery between EEGs but may instead be related to records taken over the course of years without intervention, but with some decline, as well as other examples where multiple EEGs may be taken.

Safety Features to Prevent Misuse

The portable NEST system 100 may have the safety feature system 100 that includes safety features for preventing misuse of a TMS therapy (e.g., preventing misuse of the portable NEST system 100). In general, the safety feature system 110 may prevent misuse of TMS with respect to safety limitations. TMS-type devices may require strict and effective safety features to prevent misuse (intentional or otherwise). In some examples, trials and studies may have been about thirty minute sessions with stimulation for about sixty seconds or every minute. One of the safety limitations of TMS may be that each patient may only stimulate for about five to six seconds per minute where there may be thirty cycles for the thirty-minute session. This is to prevent overheating of brain tissue with extended stimulation, which may cause some significant damage if stimulation is applied for too long. The safety feature system 110 of the portable NEST system 100 may limit stimulation to relatively low level of energy (e.g., less than about one percent of what a typical TMS device provides). This safety feature may prevent overheating of the brain tissue such that the device could be used for hours, days, or indefinitely.

The portable NEST system 100 may allow for treatment about once a day. The safety feature system 110 may provide the ability to restrict treatment to be only allowed for thirty minutes per day and each patient user may have their own unique ID. The safety feature system 110 may have a phone or mobile application that may not allow patients to use the portable NEST system 100 more than once per day (e.g., user ID may be restricted for the user ID associated with patient on the phone application). The magnets themselves may have a natural maximum energy stimulation that they can provide such that over stimulation may be avoided. In addition, the stimulation from the magnets may be weak enough such that the patient could use the portable NEST system 100 (even without the safety restrictions) for longer periods of time without any safety risk. The safety feature system 110 may provide a TMS-related work threshold test where the portable NEST system 100 may be positioned over the patient's motor cortex and pulses may be generated looking for whether the patient's thumb twitches. Then, the safety feature system 110 and the TMS treatment determination system 120 may be used to adjust the amplitude based on the results of the TMS-related work threshold test. This may be an example of the energy level that may be set for the patient (e.g., with respect to the patient user ID) when using the portable NEST system 100. In general, the portable NEST system 100 may be considered safer than most conventional TMS devices in that the portable NEST system 100 may avoid dealing with conventional TMS device-type problems such as overcharging of a capacitor causing the capacitor to explode or fail, too much current being sent, and overheating coil that may cause the coil to melt or fail to function as required. Further, the portable NEST system 100 may use the safety feature system 110 to provide additional safety precautions as described in the disclosure.

The portable NEST system 100 (e.g., in the form of a portable unit device) may include the safety feature system 110 having safety features that may prevent misuse of TMS therapy for the device. There are various examples of these safety features as described in this disclosure. Another example safety feature may be tamper proofing (e.g., tamper resistance) for the portable NEST system 100.

Electrophysiology Database and Customized TMS Treatment System—Treatment Software Application

As shown in FIG. 2C, the electrophysiology database and customized TMS treatment system 200 may include the diagnosis and treatment system 210 having treatment software application(s) 212 for providing a customized treatment software application. The electrophysiology database and customized TMS treatment system 200 may include the customized treatment software application for providing TMS determined treatments based on EEG data. The customized treatment software application may provide TMS suggested treatments to a clinician as determined for each patient such that the software application may be configured to execute these suggested treatments through engagement with each patient. The electrophysiology database and customized TMS treatment system 200 (e.g., platform type system) may also allow patients to generally engage in the process of healing their brain by using an application on their patient device(s) 302 (e.g., mobile device, phone, tablet, or other computing devices). This platform system may allow patients to engage in the process of healing their brain with a device (e.g., portable NEST system 100) with the patient device(s) 302 and find the process enjoyable.

The customized treatment software application may determine TMS treatments based on a gamification application. Gamification of the TMS treatment may use the electrophysiology database and customized TMS treatment system 200 (e.g., platform-type system) that may allow for TMS treatment to be gamified, potentially by showing patients how they can control their own brain and giving them a limited ability to observe how it works for themselves. Safety locks may be used to prevent misuse where a trained professional may not be present to manage the TMS treatment. Such a customized TMS treatment system may include an EEG system as well as may be used as part of neurofeedback, where the patient may take an active part in controlling their brainwave activity along with stimulation from the TMS treatment system (e.g., electrophysiology database and customized TMS treatment system 200).

Electrophysiology Database and Customized TMS Treatment System—Clustering Analysis

The electrophysiology database and customized TMS treatment system 200 may provide clustering analysis (e.g., using the electrophysiology analysis system 204) that may be used for diagnosis and treatment (e.g., using the diagnosis and treatment system 210). After EEG measurements may be taken, a goal may be set of determining ideal frequency for treatment but may also determine insights from clustering of EEGs or EEG data for one patient against other patients. The clustering may be k-means, KNN, shared nearest network, and the like. For example, as part of clustering process, all of the EEGs may be integrated in one place such that recurring all EEG analysis may be completed and then clustering may be run (e.g., using a preferred K-means).

Electrophysiology Database and Customized TMS Treatment System—Bursting Analysis

The electrophysiology database and customized TMS treatment system 200 may provide bursting analysis (e.g., using the electrophysiology analysis system 204) that may be used for diagnosis and treatment (e.g., using the diagnosis and treatment system 210). Analysis thresholds for identification of specific activities, such as bursts of activities, may be adjusted and normalized to the EEG as EEG information is gathered. In some examples, these bursts may be labeled and noted as objects such that these labeled burst objects may be monitored based on their behavior locally and globally in the brain which may be used as further analysis in tracking progress in response to treatment. When viewing a wave on an EEG trace, the wave may be a representative of an increase and fluctuation in charge density under the electrode. The electrophysiology database and customized TMS treatment system 200 may label bursts of EEG activity in the EEG data of each patient (e.g., labelling bursting or bursts in the EEG data). The electrophysiology database and customized TMS treatment system 200 (as part of the EEG and TMS process 400) may outline use of a continuous wavelet transform (e.g., formal tool that may provide a representation of a signal by letting scale and translation parameters of wavelets vary continuously) in order to identify a specific type of wave in EEG data. In some examples, these techniques may be used to explore and find specific types of waves that may be associated with or may relate to diagnoses (e.g., waves that may have a pattern or signature associated with seizures that may be referred to as “seizure waves”). One example use of continuous wavelet transformation may be an identification of shrinking or increasing of size of waveforms that may be taken through each channel until the electrophysiology database and customized TMS treatment system 200 may identify a representative at least a portion (e.g., wavelets) of the waveform as seizure activity. This representative portion may be referred to as a burst of activity where the tissue itself may essentially be ringing at a specific rate or frequency. In addition to utilizing the continuous wavelet transform to identify wavelets, the electrophysiology database and customized TMS treatment system 200 may also identify where there may be a repetitive solitary activity that may be related two in a row or three in a row. The electrophysiology database and customized TMS treatment system 200 may label bursts that may be noted as objects such that these object-related activities may occur locally and globally in the brain which may be used as another metric to track progress in response to treatment. This metric for burst-related objects and activities may also as be used for another level of analysis and understanding (e.g., as part of the EEG and TMS process 400). For example, other analyses related to bursts may include determination how much power may be in a band, which may be the most basic way of looking at EEG and being able to identify that there may be two hundred bursts wanted when treatment started and there may be two hundred seventy bursts preferred for analysis after a week. This may be a granular analysis where the electrophysiology database and customized TMS treatment system 200 may look for determining the strength of the band that may be moving through cortical tissue, ratio of movement of the ripple band, delay, etc. This may allow for the electrophysiology database and customized TMS treatment system 200 to have another degree of understanding of the health of the bursting activity against other patients who had pre and post treatments from the patient recorded EEGs data store 208 (e.g., database) which may continue to increase with useful data that may be used to provide interesting insights that may relate to bursting. Whenever a new measurement may be discovered, the patient recorded EEGs data store 208 may be analyzed to determine how intervention of particular treatment (e.g., specific TMS treatment parameters) may have impacted EEG data and may have adjusted the EEG data such that this particular treatment may be modeled for use by other patients who may be new patients with similar diagnoses based on EEG data. These modeled treatments may be used to change and improve metrics of the new patients. This additional granular layer and use of bursting as a measurement may allow for the electrophysiology database and customized TMS treatment system 200 to specify any frequency band of burst such as theta (or for about 7-8 Hertz).

In general, EEG activity in posterior brain regions may be important for awareness and sensory input whereas EEG activity in frontal regions may be important for other reasons such as working memory and higher function cognition. How these parts or regions of the brain (front and back) may communicate with each other may be another layer of importance that may provide interesting data related to local function, local stability of measure, global stability of measure, and why patients who may have local stability in posterior regions versus frontal regions may have differences in therapy response but may also have differences in genetic variability as a cluster. These interesting data points may be utilized by the electrophysiology database and customized TMS treatment system 200 to provide additional analyses through identification of EEG endophenotypes or signatures. In some examples, there may be typically about nine microvolts at a frontal theta where about 6.5 Hz frontal bursts (about 50 of them) may be identified. There may be some patients with about fifty frontal bursts in about 6.5 Hz, which may identify and possibly diagnose patients as having alcoholism, substance abuse, a head injury of a certain type, and the like. The electrophysiology database and customized TMS treatment system 200 may take this additional labeling and measurements versus other important clinical insight for providing diagnoses and treatments (e.g., using the diagnosis and treatment system 210). Machine learning from the artificial intelligence system 224 may be used to provide a diagnosis based on percent confidence, a measurement that may be taken as fitting into a bin or folder related to diagnoses. This may be similar to an approach where a blood test may be used to provide a relative percentage of heritability. EEG data may be used to determine a percentage of confidence that a component or portion of the EEG may have a pattern that may be related to reported symptoms and/or diagnoses. As more and more data may be obtained, the number of insights and accuracy may increase that may help add context to EEG data. The electrophysiology database and customized TMS treatment system 200 may include software that may provide questions for patients. The patients may use a mobile device (e.g., iPad) to input answers to these questions (e.g., in a survey at the clinic) before an EEG may be taken (e.g., where EEG may have labels). The electrophysiology database and customized TMS treatment system 200 may use the input answers along with the EEG data for providing diagnoses, brain health indications, and treatments for patients. In some examples, the survey of questions may be in paper form, which may be provided to patients at the clinic. The electrophysiology database and customized TMS treatment system 200 (as part of the EEG and TMS process 400) may provide the ability to scan the paper and convert the scanned document into data (e.g., may use simple optical character recognition (OCR) to identify, understand, and extract data from text in scanned forms). The answers to the questions in the paper form may be extracted by this service or tool of the electrophysiology database and customized TMS treatment system 200. This information may be used as context for the EEG data that may be captured and recorded for each patient.

The electrophysiology database and customized TMS treatment system 200 may provide intelligence testing as well (may also be called neuropsychiatric battery testing). This intelligence testing may be described as a brain check, which can be used, on tablets (e.g., five-minute psychiatric battery test). The electrophysiology database and customized TMS treatment system 200 may use this brain check or brain test providing additional information and coloring to the EEG data. These tests may include clinical scales, symptom profiles, and/or chief complaints for patients that may also be received by the electrophysiology database and customized TMS treatment system 200. The electrophysiology database and customized TMS treatment system 200 may use this information with the EEG data for providing diagnoses, brain health indications, and treatments for patients.

The electrophysiology database and customized TMS treatment system 200 may use clinically generated data (e.g., including labels) with EEG data for providing diagnoses, brain health indications, and treatments for patients. The clinically generated data may be received by the electrophysiology database and customized TMS treatment system 200 from a chief complaint for each patient. For example, where a patient may be diagnosed as ASD, the patient's data may put them into a diagnostic bin or folder and then the electrophysiology database and customized TMS treatment system 200 may provide a software layer or component that may request information and/or data from the user (e.g., from clinician user or patient user). In some examples, the requested information may be generally the same for each patient or may be specific (and may vary) based each patient's brain condition. The requested information may include clinical scales. In some examples, the requested information may be for the clinical scales to be inputted as individual questions or components of the clinical scale. The electrophysiology database and customized TMS treatment system 200 may provide this tool to clinics as a data dashboard so that clinician users may view status of patients (e.g., PTSD score for a patient decreases) and a place for users to input data that may be collected (e.g., clinical scales).

Referring now to further example implementations, FIG. 5 shows an example burst histogram 1500 for a sample EEG that may be used for determining a diagnosis of patient based on bursting characteristics. This histogram may be included in reporting (e.g., using the reporting system 206) such as an example reporting screenshot within the electrophysiology database and customized TMS treatment system 200.

Electrophysiology Database and Customized TMS Treatment System—Analysis

The electrophysiology database and customized TMS treatment system 200 may include the electrophysiology analysis system 204 for providing various analytics. The electrophysiology database and customized TMS treatment system 200 may include EEG data such that most (if not every single one) of the EEG data may have over fifteen thousand data points which may be used with relatively sophisticated discriminant analysis. For example, a variety of analyses may be applied and may be utilized in terms of discriminant analysis. One second may be two hundred data points but even those two hundred data points may provide a new relation between different data points. Thus, one second of EEG may be more than two hundred data points for various measurements and when extended to ten minutes, the data points may increase to over one hundred thousand data points (e.g., about one hundred and twenty thousand data points).

The electrophysiology database and customized TMS treatment system 200 may include an electrophysiology database system having electroencephalograms (EEGs) from pre and post treatments for providing feedback in terms of diagnosis and treatment of patients and may include the electrophysiology analysis system 204. The electrophysiology database system may apply big data analytics and/or aggregated data analytics on the EEGs (e.g., using the electrophysiology analysis system 204) for determining the diagnosis and the treatment of each patient (e.g., providing electrophysiology big data analytics). The electrophysiology database and customized TMS treatment system 200 (e.g., using the electrophysiology analysis system 204) may provide a set of aggregated data analytics on the EEG data from a set of multiple patients that may be used as an input for at least one of determining a diagnosis and recommending a treatment for the patient.

Electrophysiology Database and Customized TMS Treatment System—Analyzing EEGs with Frequency-Based Algorithms

The electrophysiology analysis system 204 of the electrophysiology database and customized TMS treatment system 200 may provide for analyzing of EEG data with frequency-based algorithms. The electrophysiology analysis system 204 may provide for discovery of frequency disparities (e.g., using the artificial intelligence system 224) that may often correlate with symptoms related to cognitive challenges (e.g., PTSD and autism). This analysis may enable both automated and human driven neurological treatment planning. The EEG and TMS process 400 may include analysis/discovery of cognitive state to treatment. For example, with an autistic patient, the EEG data may be expected to have a high density of a type of activity in the posterior of the patient's brain that may not be seen in the front. This may be a relatively reliable pattern. A patient with a head injury may be expected to have a relatively high amount of slow activity in the front compared to people who may not have head injuries. The electrophysiology database and customized TMS treatment system 200 may be built as to include a database of individual EEGs with labels that may indicate that new patients having EEGs with a relatively high amount of frontal slowing needs may relate to a normal amount of frontal slowing with a person having a concussion. This observation and/or identification may indicate the treatment that may be needed. Diagnosis may be provided based on a significant amount of data that may have statistical p-values for confidence that may allow for EEG data to be placed in a head injury folder or bucket where EEG data may have patterns associated with patients having head injuries as described in the disclosure. As the p-values improve, then it may be possible to obtain FDA approval on diagnoses. At the very least, the electrophysiology database and customized TMS treatment system 200 may be set up such that there may be these parameters and these frequency boundaries where if a peak value alpha frequency may be under about eight Hertz. Where the patient may be older, there may be some dementia that may worth observing related to Alzheimer's diseases. In this example, the electrophysiology database and customized TMS treatment system 200 may use EEG data and associated patterns of EEG data for diagnosing Alzheimer's, dementia, and/or Parkinson's disease. As the data store and/or database of the electrophysiology database and customized TMS treatment system 200 may improve in terms of quantity and quality of EEG data, there may be increases in statistical confidences such that each patient may fit into a cluster. If you combine the associated EEG data with a few clinical symptoms, the diagnosis itself may be supportive. The electrophysiology database and customized TMS treatment system 200 may use the addition of other data (e.g., clinical data) in combination to help provide context for the EEG data in making diagnosis determinations.

There may be algorithms used with historical data as part of this analysis. The electrophysiology analysis system 204 may use mathematical algorithms for identifying pertinent features. The electrophysiology analysis system 204 may involve building more with a first measurement that may be an additional measurement, which may be input variables. The electrophysiology analysis system 204 may involve weighting as part of the algorithms. Given input measurements (e.g., age of the patient), if there are measurements at about 8 Hz and another measurement at about 10 Hz, then enough information may be available to define and determine a state of the patient. For example, a decision matrix may be used with clinicians such that reliable algorithms that may be in existence may be captured in a programmatic sense. These algorithms may be based on correlations between components of EEG data that may be associated with a mental disorder (e.g., autism in children). When there is, an observation of a component that may be well correlated with what may be seen in a traumatic brain injury (TBI); the electrophysiology analysis system 204 may capture this algorithm because the input and output may be used for clustering. The electrophysiology analysis system 204 may use an algorithm that may include weighting variables by location that may output a number value for treatment. These weights may be adjusted for intermediate steps to make sure outputted number values may be reliable.

The electrophysiology analysis system 204 may use technology for analyzing a cognitive state (e.g., use the artificial intelligence system 224). The electrophysiology analysis system 204 may analyze EEG data and may derive deep insight into each client's brain health such that the analysis may combine expertly honed algorithms with historical data (e.g., years of data) to create an actionable cognitive report (e.g., using the reporting system 206). For example, identification of principal EEG spectral components and identification of activities, by region, may form the baseline EEG profile that may inform therapy approach and expected responses. With this analysis, frequency disparities may be discovered by the electrophysiology analysis system 204 that may often correlate with symptoms related to cognitive challenges (e.g., PTSD and Autism). The electrophysiology analysis system 204 (e.g., using the artificial intelligence system 224) to understand these disparities may help patients and clinicians in determining the best strategy in terms of current and future treatment.

The expertly honed algorithms may be based on a variety of factors and variables. For example, included in the algorithms may be rolling windows that may adaptively scale to capture and classify specific wavelets versus background noise, with a possibility for integration of wavelet transforms during preprocessing for further optimization. At this stage in the process, wavelet validity may be tested with nearest-neighbor coherence and transfer entropy measures counted above preset thresholds. Relative EEG power distribution may be determined from identified wavelet ‘objects’ distribution and identification of principal EEG components that may contribute to the generation of the EEG profile, or ‘type’, which may be connected to sensitivity or resistance to therapeutic response, thus triggering adjustments to therapy. Some EEG types may include low voltage cases requiring multiple locations of stimulation, or focal slowing due to stroke that may require local and lateralized stimulation based on identification of functional deficit foci, or identification of EEGs via burst analysis where bursts may remain scattered after the first few EEGs that may require additional treatment locations. Identification of multiple alpha cortical generators may be more resilient to therapy and may require posterior stimulation over anterior stimulation.

As it pertains to cognitive reports, specificity may be brought through the identification of patient ‘brain type’ via specific predetermined characteristics. In addition, machine-learning techniques may be applied such as k-means clustering and principal component analysis as well as definition by a specific variable with predefined measurement cutoffs. For example, an anterior-posterior alpha band amplitude ratio may be combined with a regional frontal coherence threshold for identification of patients with frontal alpha generation. Given the identification of EEG subtype, patients may be compared to an average representative of similar brain types, as collected by the EEG system (e.g., as part of the EEG and TMS process 400). This relative comparison may provide a relatively more accurate measure of individual ‘cognitive network’ health versus a comparison of the patient versus an average from all EEG profiles.

Additional insight into the network health and subsequent cognitive state may be through an application of a relative brain synchrony index (RBSI) where an ideal frequency of function may be identified. The identified ideal frequency of function may differ from peak alpha frequency and a comparison between current peak alpha frequencies versus ideal frequency may help gauge an overall reduction in function versus ideal. Therefore, cognitive capacity versus individual ideal in addition to capacity for response to treatment and possibly overall duration of treatment may be determined. Through utilization of adaptive peak identification algorithms, a patient may have a dominant EEG alpha at about 8.7 Hz, with a reduced but present alpha at about 9.8 Hz, and at about 10.6 Hz. This analysis may indicate a conclusion that the patient may be about two degrees from ideal peak alpha frequency and therefore working at relatively reduced capacity and prime for response to neuromodulation. A patient may have a dominant spectral frequency of about 7.5 Hz frontally, and a posterior dominant alpha rhythm at about 8.5 Hz, with evidence of rhythmic alpha bursting at about 9.3 Hz and about 10.2 Hz. This patient may be considered more severely impacted clinically due to the reduction of spectral activity below the cutoff for alpha activity (e.g., about 8.0 Hz cutoff) and due to a separation from what may be ideal peak frequency of about four or more rhythmic EEG steps.

The EEG and TMS process 400 may utilize other types of algorithms. The EEG and TMS process 400 may utilize Hilbert transforms, hamming windows, and/or different variations on mother wavelets (e.g., Daubechies (db4), Morlet, etc.). In some examples, progressively narrowing band pass filters may be used around detected peak frequencies. For example, if a peak frequency of about 11 Hz is noted, there may be multiple ordered filters, such that a first order may be from zero (0) to twice a detected frequency, the second order may be a set percentage from zero (0) and the detected frequency and correspondingly reflected on the faster range, etc. These progressive filters may indicate cortical organization by their ratios, or relative network health by their interrelations.

In terms of data depth, frequency may be particularly focused on for analysis. The electrophysiology analysis system 204 may analyze for following informative data: relative burstiness and amplitude of a burst, how many wavelets may be in a burst, how each burst may move through space, etc. This informative data may be additional input measurements used by the electrophysiology database and customized TMS treatment system 200 (e.g., for analysis by the electrophysiology analysis system 204 and for diagnosis and treatment by the diagnosis and treatment system 210).

When different regions of the brain may share a same wave pattern, the brain may be more efficient. When regions are not as synchronized, cognitive symptoms may be observed. For example, when a client has depression, their alpha and theta wave patterns may typically differ across left and right frontal hemispheres. The electrophysiology database and customized TMS treatment system 200 may provide for collection of historical data that may allow for correlation of unique patterns to symptoms and mental conditions.

Electrophysiology Database and Customized TMS Treatment System—Predictive Analytics

The electrophysiology database and customized TMS treatment system 200 may include the predictive analytics system 222 that may be used to identify people before they get ill and may provide treatment and/or care recommendations based on the electrophysiology database and customized TMS treatment system 200 (e.g., specifically patient recorded EEGs data store 208). In some examples, insights may be obtained from multiple patients for predicting what may be triggering mental disorder (e.g., not enough sunlight for depression). In some examples, EEG frequency content, distributions, and different bands for patients who may be healthy and may have no disruptions may be obtained such that there may be different normal resting EEG patterns and densities (e.g., as relating to different brain types). In some examples, when EEGs may be measured for patients with their eyes closed, the EEG recorded data may have an excess of beta activity or working brain function whereas most patients may have lower density beta EEG data during eyes closed. The electrophysiology database and customized TMS treatment system 200 may identify this beta activity and indicate possibility for anxiety disorders. By measuring this relevant data, the diagnosis and treatment system 210 may identify patients as having anxiety (e.g., patient may be predisposed to anxiety). With regard to some of the EEG patterns observed, the diagnosis and treatment system 210 may look to increases in low frequency EEG activity during eyes-closed resting states and compare this data versus what may be expected for the same condition and similar populations. The electrophysiology database and customized TMS treatment system 200 may identify a density of slow-wave activity as abnormally high and may indicate a possibility for cognitive slowing or attention interference that may be corrected. The RBSI may be applied to indicate when dominant spectral rhythms globally may be slowing as compared to what may be considered ideal for that patient, and may indicate that cognition is declining and treatment may be warranted. There may be an ideal EEG type of template that may be used as a reference for determining treatment (e.g., by the diagnosis and treatment system 210). In some examples, metrics or patterns may imply positive or negative.

In general, different brain types may have pros and cons (e.g., strengths and weaknesses) which may be due to resting rhythms contributing to either more or less sympathetically activated neuronal systems (e.g., parasympathetically). Some brain types may be better at focusing in on single details than others. There may be personality and performance characteristics associated with different brain types. In terms of performing treatment, the diagnosis and treatment system 210 may determine and suggest treatments that may try to lower theta power, try to change beta power, try to adjust other parameters, etc. These parameter improvements may relate to achieving idyllic EEG data. This may be where clustering may be used by the diagnosis and treatment system 210 (e.g., also using the electrophysiology analysis system 204). For example, if the diagnosis and treatment system 210 identifies a patient's brain type, the diagnosis and treatment system 210 may then compare the patient's brain to other patients with the same brain type (e.g., using the electrophysiology analysis system 204) and the diagnosis and treatment system 210 may determine to what degree there may be an addition of activities as disruptions. Then, the diagnosis and treatment system 210 may indicate that there may be a need to address the activities to bring brain status to a relative point that may be ideal for the patient. From a predictive analytics perspective, the predictive analytics system 222 may expect specific brain types to have predispositions towards different mental disorders or to be more sympathetically or parasympathetically activated such that the diagnosis and treatment system 210 (e.g., using the predictive analytics system 222) may provide feedback and then the additional perspective of frequency may be added (e.g., comparing slow frequency to where the frequency ought to be, as an ideal from which departure may signal a warning flag).

The electrophysiology database and customized TMS treatment system 200 may determine from EEG data that some people may have a relatively higher rate of depression given that these people may not have enough sunlight (e.g., using big data analytics and/or aggregated data analytics with predictive analytics). Thus, the electrophysiology database and customized TMS treatment system 200 may want to provide an outreach to promote certain types of wellness behaviors. Further, the predictive analytics system 222 of the electrophysiology database and customized TMS treatment system 200 for predicting what may be triggering a mental disorder (e.g., not enough sunlight for depression). The predictive analytics system 222 may consider outside external factors for determining what may be triggering specific mental disorders in addition to the mental disorders themselves. The predictive analytics system 222 may monitor at how data may be handled such that the more information available may provide context to the data, which may be used to make determinations from the data. Information related to context may include disrupted sleep, limited sunlight, history of head injury, opiate use or drug use of any kind, a stroke, etc. This may provide context on why different factors in the EEG data may not normally be there and why other external factors that may relate to a clinical biography of the patients may be affecting EEG data results. In general, there may be brain functions that may be disruptions from ideal function and there may be ideal function. Having an extended period without appropriate morning sunlight and disrupted sleep may reinforce that disruption. In some examples, too much of that disruption may push patients towards what may be considered and classified as a mental disorder or a disruption in the normal brain function to the extent where it may cross some clinical boundary. The electrophysiology database and customized TMS treatment system 200 may have an algorithm that may take as input, for example, living in places such as Seattle or Norway may have limited sunlight for half the year, or a veteran or an active duty person may suffer from shell shock and other forms of post-traumatic stress disorders. This type of information may be provided to trained clinicians, which may be provided as input when interpreting the EEG data. The electrophysiology database and customized TMS treatment system 200 as a platform that may have these types of data fields as input against which these types of analysis may be conducted. The electrophysiology database and customized TMS treatment system 200 may obtain contextual information associated with the EEG data useful for analysis and making decisions by various systems and components (e.g., used by the electrophysiology analysis system 204, the reporting system 206, the diagnosis and treatment system 210, the treatment modelling system 220, the predictive analytics system 222, and the artificial intelligence system 224).

The electrophysiology database and customized TMS treatment system 200 may allow for inputting from a data stream view (e.g., data that may be coming from clinician meetings may be inputted to the system 200). There may be a patient registration. If the patient shows up at any clinic, the patient may be registered and there may be a chief complaint field that may be filled out and submitted to the electrophysiology database and customized TMS treatment system 200 for the patient. The chief complaint field may include information such as the patient may have autism, the patient may have a history of head injuries, or the patient may have anxiety. The chief complaint may also include date of birth, gender, and other biographical information, which may be received from the patient information field. The clinical data that the clinician supplies to the electrophysiology database and customized TMS treatment system 200 may include clinical data that may be gathered. The electrophysiology database and customized TMS treatment system 200 may request for the data after the clinician meeting (e.g., when reviews may be completed). The electrophysiology database and customized TMS treatment system 200 may be built as a software infrastructure that may automatically capture as a front end as a service for clinicians. For example, use of the electrophysiology database and customized TMS treatment system 200 such that patients may be able to have clinical scales easily accessible which may provide additional insight on the EEG data. This associated information may be tracked and tagged with EEG data appropriately.

The electrophysiology database and customized TMS treatment system 200 (specifically the predictive analytics system 222) may utilize prodromal or predictive medicine where populations may identify in a preventative manner based on EEG data before they develop (e.g., using big data analytics and/or aggregated data analytics with predictive analytics). The predictive analytics system 222 may identify and outreach to higher health risk populations based on EEG data in a database and may encourage the patients to seek medical care depending on studies (e.g., there may be a higher rate of cervical and dermatologic cancers in one region over other regions in the same country that may also be associated with higher rates of depression). The predictive analytics system 222 may identify and outreach to populations in a preventative manner before the patients may develop cancer or heart disease based on EEG data in the electrophysiology databases (e.g., of the electrophysiology database and customized TMS treatment system 200). The predictive analytics system 222 may use data and information (e.g., including contextual information) in the electrophysiology database and customized TMS treatment system 200 to identify patients before they develop, for example, dementia or Alzheimer's disease. In addition, the predictive analytics system 222 may use EEG data (e.g., electrophysiology databases of the electrophysiology database and customized TMS treatment system 200) to identify patients who may have a certain type of fragility (e.g., more prone to PTSD, which may include applications for military, police officers, firefighters, etc.). The predictive analytics system 222 may identify populations and/or patient groups based on EEG data and other information in the electrophysiology database and customized TMS treatment system 200 for providing insights and/or predictions for these identified populations and/or patient groups.

There may be disruptions that may normally develop and may increase. These disruptions may be related and/or associated with brain types as described in the disclosure. There may be different bands of brain activity such as from slow to fast, delta theta on the slow side of the brain, alpha beta and some gamma on the faster side of the brain, etc. In some examples, patients may have more beta activity than other patients. Brains may have high amounts of beta activity. It may be normal to have some beta activity but there may be heterogeneity in the population such that some patients may be predisposed towards having more beta activity. There may be more alpha or theta or different densities in these relationships. During resting state, where some patients may be observed to have more elevated beta activity compared to expected norms, these patients may have a predisposition towards anxiety, feeling restless, or being more sympathetically activated, which may push patients towards an anxiety class of disorders. The electrophysiology database and customized TMS treatment system 200 may provide a prediction that assess how well this anxiety may be kept in check. For example, whether kept in check by the density of different brain rhythms, which may be in the alpha, range (brain clock rhythm) or may be kept in check when it comes to lifestyle habits. The electrophysiology database and customized TMS treatment system 200 may provide a brain score or a brain metric that may score how much beta or restlessness patients may have which may be used against a daily log to determine if the patient may be at risk. The patient may be more at risk for anxiety disorders or PTSD than patients who may not have high amounts of beta activity. This type of correlation may relate to any of the anxiety classes.

There may be patients that may have elevated alpha rhythms when eyes closed (e.g., higher density and in different regions than where it may be expected). Some patients may generate these alpha rhythms frontally whereas a majority of patients may generate these rhythms posteriorly. In some examples, the electrophysiology database and customized TMS treatment system 200 may notice from notes or other information that patients may have too many parasympathetic behaviors/responses (e.g., “rest and digest”), too little sympathetic behaviors/responses (e.g., “fight or flight”), and relatively low activated brains may be diagnosed as having depression or low energy/mood. From this information and/or data, the electrophysiology database and customized TMS treatment system 200 may cluster to provide an indication as to the patient being predisposed.

The electrophysiology database and customized TMS treatment system 200 may monitor and provide analysis for relative brain synchrony (RBS) size (e.g., this may be a shortened version of the relative brain synchrony index (RBSI)) where the electrophysiology database and customized TMS treatment system 200 may refer to frequency or speed of the brain clock. The frequency or speed of brain clock may be referred to with respect to therapy protocols. The electrophysiology database and customized TMS treatment system 200 may choose a frequency or may calculate the frequency. This frequency may be typically in the alpha range. The frequency may not be in the alpha range because of the brain clock activity. As patients age and grow, the patient's may speed up to a certain frequency and may slow down normally with time. People's frequency or speed may typically slow down eventually.

The electrophysiology database and customized TMS treatment system 200 may check a degree to which a patient may be slowing down relative to ideal for the patient. With an ideal frequency, the electrophysiology database and customized TMS treatment system 200 may calculate a patient is zero point. Interesting insights may be discovered such as the patient may tend to slow down in about 0.8 to about 1.2 Hz jumps that may seem to correspond with heart rate frequency. If a patient may be at about 11.3 Hz, the electrophysiology database and customized TMS treatment system 200 may observe activity at about 10.2 Hz and about 9.1 Hz, for example. If a patient may be at about 9.6 Hz, the electrophysiology database and customized TMS treatment system 200 may observe frequency over 10.5 Hz. This may correspond to notes such as if the patient may be at about 11.3 Hz, the patient may be at zero point. In addition, the electrophysiology database and customized TMS treatment system 200 may determine 11.3 Hz as a posterior rhythm that may occur about twenty percent of the time. In the same region, the electrophysiology database and customized TMS treatment system 200 may observe a posterior rhythm at about 10.1 or about 10.2 Hz that may occur about sixty percent of the time. In some examples, the electrophysiology database and customized TMS treatment system 200 may make the postulation that the patient should be up at about 11.3 Hs instead of being down at about 10.2 Hz. This may be one note or an F1 difference from ideal. The greater the differential may be, if down two notes slower or three notes slower, the more disruptive the patient may be from ideal and the more at risk the patient may be may which may lower the patient's relative brain score. When the frequency is about eight or sub-eight Hz frequencies, there may be possible memory loss or memory disruption, elevated fatigue, and other symptoms that may be seen more commonly in Alzheimer's or Parkinson's patients. If patients have a major traumatic brain injury, the patients may immediately move from either F01 down to an F4 or F5. If the patient has a stroke, it may be quite traumatic and may be relatively difficult for the patient's brain to organically move itself back up in any short or relative period of time without any intervention. The electrophysiology database and customized TMS treatment system 200 may be used to analyze alpha rhythms such that the fast Fourier transform (FFT) may look at frequency, speed, or number of oscillations per second. For younger patients, frequency may be typically faster whereas with older patients, frequency may be slower. This may relate to power increases (e.g., power may drop and may then increase again) where amplitude may decrease and may resynchronize faster. In general, patients with faster brains may have improved working memory (e.g., this may be based on lining up of alpha peak frequency versus working memory showing positive correlation). In some examples, the electrophysiology database and customized TMS treatment system 200 may determine a projected ideal speed for each patient. This ideal speed or rhythm may be generated posteriorly but may occur globally.

The electrophysiology database and customized TMS treatment system 200 may observe on the development delay side that the patient may have a developmental delay vs. autism. The electrophysiology database and customized TMS treatment system 200 may make discriminations between EEG data and clinic reviews for providing indications of diagnosis. There may be kid patients that may be listed as being diagnosed with autism but from analysis of EEG data and other information, the electrophysiology database and customized TMS treatment system 200 may determine that the kid patients may have developmental delays not autism. The characteristics on the symptom profile may be similar between autism and developmental delay (e.g., delayed language sensory processing awareness). EEG data and other tests (e.g., genetic testing) may assist the electrophysiology database and customized TMS treatment system 200 in distinguishing diagnosis between autism and developmental delay.

The electrophysiology database and customized TMS treatment system 200 may be used with a patient having a head injury (e.g., concussion). The patient may present classic concussion syndromes and the patient's EEG data may demonstrate focal slowing in the theta range both at an injury site and possibly in other sites correspondent to the injury. Neuromodulation may be recommended and applied for about two weeks, which improves the patient's brain status and score(s). In some examples, neuromodulation may be applied at about 10.3 Hz. After applying the stimulation, there may be entrainment because of stimulation and resultant of about 10.3 Hz may be observed where previous slowing occurred. When the magnetic field may be applied at a repetitive rate, the magnetic field may produce neural entrainment that induces charge locally and influences the firing rates of underlying cortical tissue and local field potential. The resultant entrainment may affect resting network function such that identified focal theta may dissipate with treatment and the entrained frequency emerges.

The electrophysiology database and customized TMS treatment system 200 may suggest a few strategies in treating (e.g., may be executed and/or implemented by the portable NEST system 100). One example may be a reinforcement of ideal function local to the site of generation. Another example may be treating function where it may be at the greatest deficit. A combination of both may be recommended and executed.

For applications involving stroke, the electrophysiology database and customized TMS treatment system 200 (and specifically the diagnosis and treatment system 210) may determine treatment for the stroke patient. For example, where the stroke patient may have an injury on their left side, the electrophysiology database and customized TMS treatment system 200 may recommend treatment on the right for the stroke. The electrophysiology database and customized TMS treatment system 200 may recommend treatment of the injured tissue (e.g., based on the identification of an injured area of the brain). The injured region may be shown to have a slowing effect especially compared to non-injured regions of the brain. The electrophysiology database and customized TMS treatment system 200 may discriminate between these regions as far as what may be injured tissue or tissue slowed down by several notes. The diagnosis and treatment system 210 may determine and suggest target treatment based on the brain's function.

The electrophysiology database and customized TMS treatment system 200 may include the electrophysiology database system that may apply big data analytics and/or aggregated data analytics on the EEGs (e.g., using the predictive analytics system 222, the electrophysiology analysis system 204, and/or the diagnosis and treatment system 210 in any combination) for determining the diagnosis and the treatment of each patient (e.g., providing electrophysiology big data analytics and/or aggregated data analytics) with respect to predictive analytics. The electrophysiology database system (e.g., using the predictive analytics system 222) may generate predictive analytics based on the big data analytics and/or aggregated data analytics that may be used to identify patients and provide appropriate treatment for the identified patients based on the EEGs (e.g., EEG data). In some examples, the electrophysiology database and customized TMS treatment system 200 may generate predictive analytics based on the aggregated data analytics (e.g., using the predictive analytics system 222) that may be used to identify patients and provide appropriate treatment for the identified patients based on the EEG data.

Electrophysiology Database and Customized TMS Treatment System—Stem Cell Treatment Recommendations

There may be a stem cell treatment process that may include applying stem cells to improve connectivity of neurons in the brain of a patient. As described in the disclosure, this stem cell treatment process may be recommended by the electrophysiology database and customized TMS treatment system 200. Use of the stem cell treatment process may be part of the EEG and TMS process 400. In further examples, the stem cell treatment process may be part of the portable NEST system 100 and/or the electrophysiology database and customized TMS treatment system 200 in any combination. The stem cell treatment process may be used by patients where connectivity may not be present or possible for existing neurons and networks. The stem cell treatment process may be used to create new neurons where there may be a lack of neurons in terms of connectivity. The stem cell treatment process may be used to enhance stem cells. The stem cell treatment process may adjust one or more parameters based on feedback of gene expression for the neurons.

The electrophysiology database and customized TMS treatment system 200 may recommend stem cell treatment. This type of treatment for the patient may be simulated (e.g., by using the treatment modelling system 220). Stem cell treatment may relate to improving connectivity with existing neurons where the connectivity may be damaged. Even with new neurons, connectivity may still need improvement. This treatment may be used to enhance stem cells.

The stem cell treatment (e.g., as a specific suggestion by the electrophysiology database and customized TMS treatment system 200) may be gene expression. In some examples, TMS may be utilized in combination with stem cells. There may be a combination effect such that stem cells may be activated locally with stimulation. Depending on the stimulation delivered to in-vitro tissue, such as delivering one set of parameters with different frequencies and timings of stimulation versus another set of parameters with the same or different frequencies and timings of stimulation in different blocks, there may be resultant differential gene expression. There may be a change depending on frequency and dose parameters behind a stimulation.

There may be other approaches with or without TMS that may result in different gene expression for each separate approach. Determining the approach to stem cell treatment may involve honing in such as through appropriate testing in a laboratory on a specific protocol that may be helpful for a specific disorder that may have a disruptive gene expression. Each treatment may be determined and may be provided specific to each treatment profile (e.g., relating to each patient and/or each disorder/condition).

The stem cell treatment may involve CRISPR gene editing (e.g., CRISPR modifications). There may be a specific treatment that may be preferred for application and the patient may not be genetically predisposed to it such that there may be an opportunity for a gene expression CRISPR combination. For example, it may be determined that specific tissues may be causing seizures because the tissues may be genetically different. EEG data may be used to focalize seizure activity and then CRISPR may be utilized to treat and attempt to repair functions of the tissues needing repair. This may be another intervention approach.

Electrophysiology Database and Customized TMS Treatment System—Other Treatment Recommendations

The electrophysiology database and customized TMS treatment system 200 may provide diagnosis and treatments (e.g., using the diagnosis and treatment system 210) that may be related to neuro-transmitter density changes. For example, if the electrophysiology database and customized TMS treatment system 200 may determine that there may be an increase in serotonergic expression, a recommended treatment may be to lower selective serotonin re-uptake inhibitors in combination with treatment as an intervention. Other treatments may include suggested medications that may be made more powerful by utilizing modulation in different ways to reinforce and change gene expression to make it such that use of medication to lower dosage to achieve the same response or results that may not be achieved at any dosage.

The electrophysiology database and customized TMS treatment system 200 may also suggest medications such as anti-depressants for use with or without the portable NEST system 100. It has been shown that anti-depressants may have one or more neurogenic properties. Neurons may be created in the brain. However, these created neurons may not be connected effectively to other neurons. One of the advantages of the described targeted TMS therapy is that it may increase neuronal connectivity. Further, the targeted TMS therapy may be combined with anti-depressants (e.g., selective serotonin reuptake inhibitors (SSRIs), serotonin and norepinephrine reuptake inhibitors (SNRIs), etc.) to provide improved results in treating patients.

Electrophysiology Database and Customized TMS Treatment System—Modelling Treatments

The electrophysiology database and customized TMS treatment system 200 may use the treatment modelling system 220 to apply the electrophysiology database (e.g., Big Data from the patient recorded EEGs data store 208) to model treatments. The electrophysiology database and customized TMS treatment system 200 may include the electrophysiology database system that may apply big data analytics and/or aggregated data analytics on the EEGs (e.g., using the treatment modelling system 220, the electrophysiology analysis system 204, and/or the diagnosis and treatment system 210 in any combination) for determining the diagnosis and the treatment of each patient (e.g., providing electrophysiology big data analytics and/or aggregated data analytics) with respect to modelling treatments. The electrophysiology database system may apply the big data analytics and/or aggregated data analytics to model various treatments (e.g., using the treatment modelling system 220) in terms of predicted impacts to the patients and results of the treatments. In some examples, the treatment modelling system 220 may also utilize the predictive analytics system 222 in combination for providing predictive analytics of the EEG data and other data as described in the disclosure.

This may include model simulations of treatments before applied to patients (e.g., based on types of stimulation treatments, frequencies, location, etc.). In some examples, big data analytics and/or aggregated data analytics may be applied in order to model impacts of specific theta-burst stimulation (TBS) treatments and general TMS treatments (e.g., using the treatment modelling system 220). The treatment modelling system 220 may facilitate large-scale experiments with collection of substantial quantities of data. These large-scale experiments may try different factors in the application of TMS treatments, such as variation in the type and nature of the TMS device, alteration in magnet positions, variability in different energy levels, etc. The treatment modelling system 220 may provide for this data to be collected into centralized or decentralized database of most (if not all) experiments. The treatment modelling system 220 may use a depth of data that may allow for discovery of other potential experiments and for a creation of models that may predict likely outcomes of other dependent variables (e.g., predictions on usage patterns of different demographics without having actually conducted experiments on the different demographics or patient types). The treatment modelling system 220 may use adjustable factors such as electrode position, energy levels, duration of treatment, and similar patient responses as part of modelling for best treatment results for each patient such that these factors may be adjusted accordingly for each patient.

The treatment modelling system 220 may be used to look at new patients coming in that may be compared to other patients who may have also come in compared to treatments that current and old patients may have received. The treatment modelling system 220 may determine for those who may have received the treatments that are similar to those who are coming in, what may be the best treatment for them and adjust treatments accordingly.

The electrophysiology database and customized TMS treatment system 200 may be used to look at areas that may be dysfunctional in treating some patients (e.g., using the treatment modelling system 220). This may relate to areas that may make ideal function and reinforcing the function. Treating autism may produce a relatively large amount of posterior alpha frequencies and a relatively small amount of frontal alpha frequencies. Initially, superior frontal function was treated, as the frontal lobe may be where there may be a deficit, which may be treated. Then, when looking into circuitry, it was found that prefrontal might have better anterior to posterior connectivity than superior frontal stimulation or superior frontal circuitry. Treatment of the cortical tissue frontally may be adjusted accordingly.

In EEG data, the electrophysiology database and customized TMS treatment system 200 may have corroborative evidence that after adding posterior location and reinforcing posterior function, the reinforcement of global corresponding oscillations may be facilitated and observed. There may be a propagation of activity from posterior to anterior regions. For some patients, addition of a posterior location may facilitate function globally too quickly for safe clinical practice. Thus, when taking a sequential EEG, the electrophysiology database and customized TMS treatment system 200 may observe the degree to which this dysfunction of interest may be shifting in density and presence and then the treatment recommendation (e.g., execution by the portable NEST system 100) may be adjusted in terms of location, frequency, and other parameters in order to reinforce function, both from the source of function and where the function is observed to be at deficit or may have disruption.

EEG and TMS Process

The EEG and TMS process 400 may provide personalized medicine including safety protocols. The EEG and TMS process 400 may be tailored to teach clinicians and may relate to design for protocols. The EEG and TMS process 400 may use a matrix in terms of the decision tree behind what may or may not be applied in terms of stimulation in different regions based on data. The EEG and TMS process 400 may be designed with data pipelines and analysis pipelines, where if the input may be ASD or developmental delay, the EEG and TMS process 400 may open up stimulation positions (e.g., coil position or magnet position). Each patient parameter may change how analysis of the data may be performed to accommodate EEG data, via different analysis pipelines. With a PTSD patient, the EEG and TMS process 400 may shut off some regional treatment initially and may reactivate the treatment after a follow up EEG is taken after treatment may be observed. The EEG and TMS process 400 may suggest a certain number of treatments with additional analysis and then add suggested treatment locations accordingly. These treatment decisions may change from mental disorder to mental disorder or mental condition to mental condition or brain injury to brain injury or substance use disorder to substance use disorder (e.g., treatment for dementia or Alzheimer's where it may not be expected to be able to push frequency as fast if the patient did not have dementia or Alzheimer's). These different diagnoses may relate to each of these data pipelines and treatment pipelines that the EEG and TMS process 400 may be able to use for recommending and providing treatment.

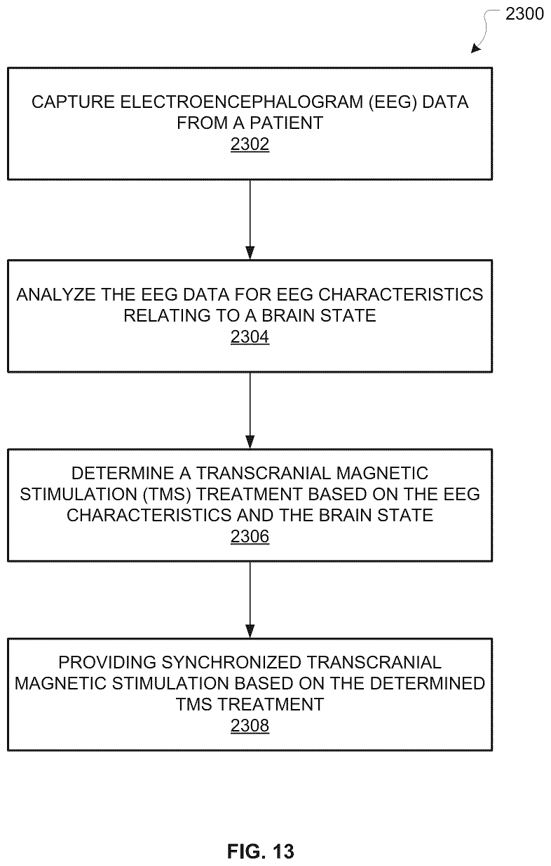

Referring now to an example implementation, FIG. 13 shows an example flowchart for a process 2300 in using EEG data to provide TMS treatment of a patient. The process 2300 may be at least a portion of the EEG and TMS process 400. For example, at least a portion of the EEG and TMS process 400 may capture EEG data from a patient at 2302. The EEG data may be analyzed for EEG characteristics relating to a brain state of the patient at 2304. A TMS treatment may be determined based on the analyzed EEG characteristics and the brain state at 2306. Synchronized transcranial magnetic stimulation may be provided based on the determined TMS treatment at 2308. In some examples, the EEG and TMS process 400 may further include comparing the EEG data analysis against EEG data modelling for the patient. The EEG and TMS process 400 may include reporting of the brain state as a brain health indicator for the patient based on the EEG data. In some examples, the EEG and TMS process 400 may include providing a diagnosis for the patient based on the EEG data. The EEG and TMS process 400 may include recapturing EEG data from the patient and redetermining the TMS treatment based on the recaptured EEG data. In some examples, the EEG and TMS process 400 may include providing other neuromodulation based on the EEG data.

Intersections of EEG and TMS Therapy

A therapy system may provide a combination of EEG and TMS treatment for patients. In examples, use of the therapy system may be part of the EEG and TMS process 400. In further examples, the therapy system may be part of the portable NEST system 100 and/or the electrophysiology database and customized TMS treatment system 200 in any combination. In some examples, the treatment may include timing of pulses related to one or more EEG waveforms and one or more other factors. The treatment may include administering of pulses using stochastic resonance. For example, pulses may not only be administered at the intrinsic frequency, but stochastic resonance may alternatively be used. This type of therapy treatment may include varying pulse frequency based on EEG characteristics and other characteristics.

Closed Loop EEG and TMS System

A closed loop system (e.g., closed loop EEG and TMS system) may provide a combination of EEG recording and TMS treatment for patients such that the system may monitor treatment and EEG recordings for adjusting related parameters based on this monitoring. In examples, use of the closed loop system may be part of the EEG and TMS process 400. In further examples, the closed loop system may be part of the portable NEST system 100 and/or the electrophysiology database and customized TMS treatment system 200 in any combination. For example, EEG recording may occur while stimulating such that there may be monitoring on how the closed loop system may respond to the stimulation such that parameters may be changed accordingly. For example, EEG may be the biometric and TMS may be the force that may be adjusted (e.g., dialing down TMS force). There may be overlapping biometric measurements and overlapping forces while the core may be EEG and TMS time periods. With this core, additional biometric measurements and additional forces may be used for neuromodulation as may be beneficial. The closed-loop system may use artificial intelligence (AI) modelling for adjusting the related parameters.

Examples of Uses of Systems and Processes

There may be a variety of examples of uses and use cases involving the EEG and TMS process 400, the portable NEST system 100, and the electrophysiology database and customized TMS treatment system 200. These systems and processes may be used in a clinic-to-clinic basis. (e.g., TMS clinics). These clinics themselves may have TMS systems (e.g., portable NEST system 100) that may be using the same protocols and mechanisms described in the disclosure. These systems and processes may be widely accepted and used by the medical community such as at doctors' offices and hospitals.

Users may engage in one or more use sessions under the disclosed methods. For each use session, data for the user for each session is stored. Data may be compared from one or more use sessions to determine a treatment level of a use session.

Referring now to example implementations, FIGS. 5-12 illustrate various analysis and reporting screen shots that may be generated based on EEG-related data. FIG. 5 shows an example burst histogram 1500 report for a sample EEG that may be used for determining diagnosis based on bursting characteristics The report illustrates low, normal and high amplitude types, count (ranging 0.0 to 4.0) over a frequency (Hz) of 8-12.

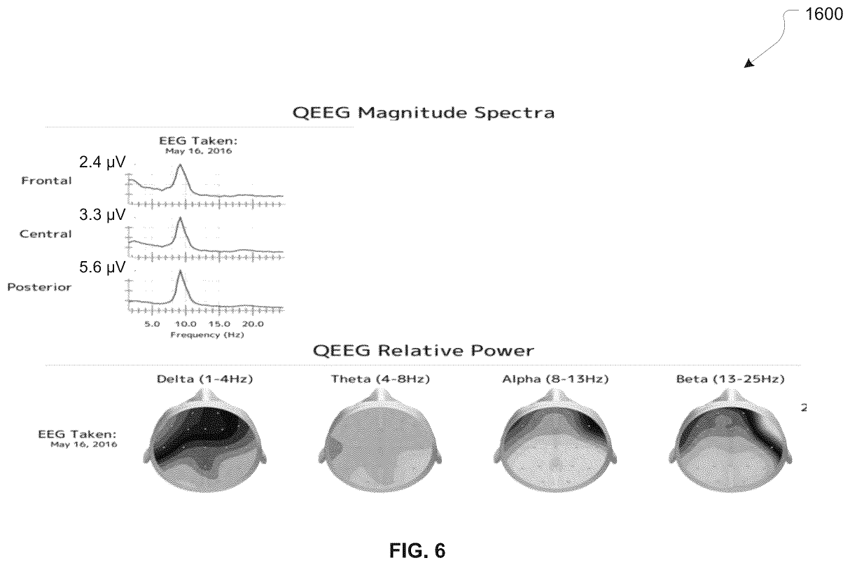

FIGS. 6-12 show brain health indicators in the form of reports including quantitative electroencephalogram (QEEG) magnitude spectra and QEEG relative power reporting for various conditions and mental disorders. These example reports may provide brain health indicators, diagnoses, and EEG data related information.

FIG. 6 shows a report 1600 that may indicate normal brain health, for example for a 45 year old male without presentation of psychiatric, physiological or neurocognitive issues. A QEEG magnitude spectra shows a frontal (peak ˜2.4 μV), central (peak ˜3.3 μV) and posterior (peak ˜5.6 μV) EEG over 0.0 to 25.0 frequency (Hz) is shown. QEEG relative power is also shown for delta (1-4 Hz), theta (4-8 Hz), alpha (8-13 Hz), beta (13-25 Hz).

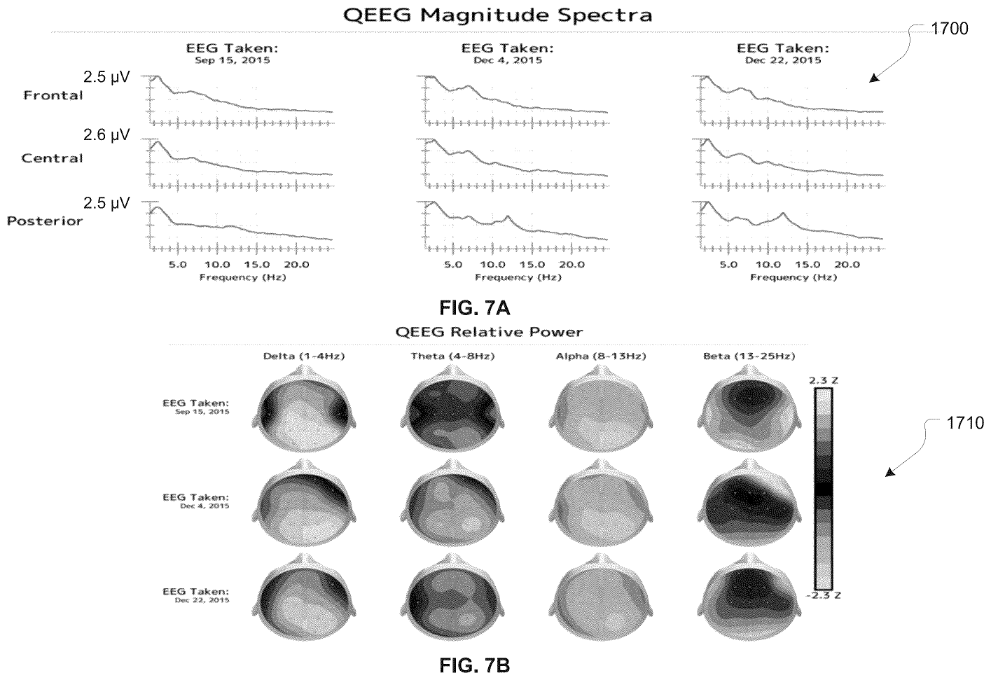

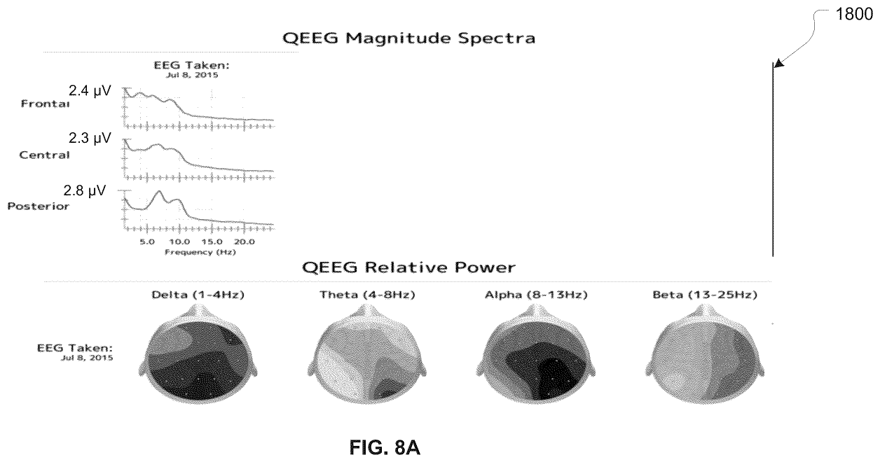

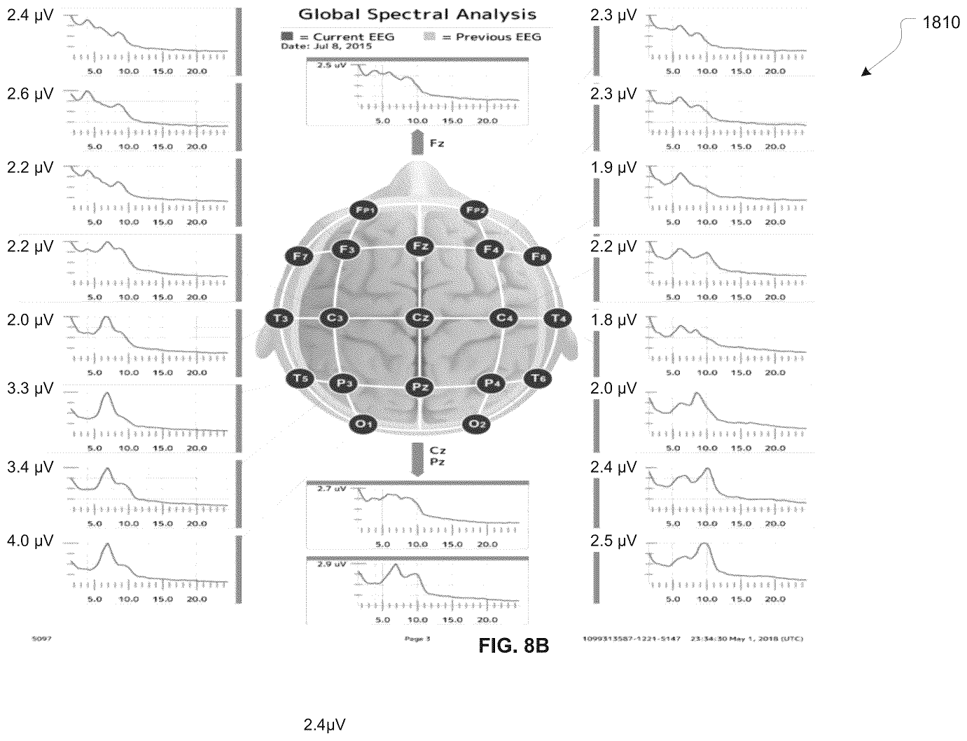

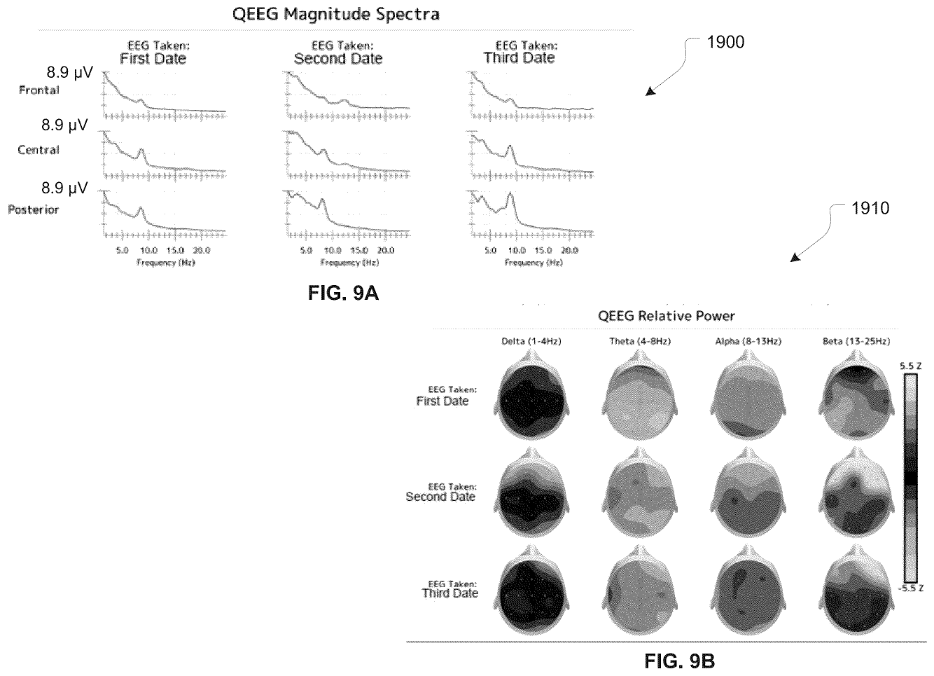

FIGS. 7A-B show a report 1700, 1710 for a 31 year old female with polysubstance use and abuse, anxiety, and panic attacks over 32 total MeRT sessions. A QEEG magnitude spectra shows a frontal (peak ˜2.5 μV), central (peak ˜2.6 μV) and posterior (peak ˜2.5 μV) EEG over 0.0 to 25.0 frequency (Hz) taken at a first date, a second date and third date is shown. QEEG relative power is also shown for delta (1-4 Hz), theta (4-8 Hz), alpha (8-13 Hz), beta (13-25 Hz) taken at a first date, a second date and a third date with a range of 2.3 Z to −2.3 Z.

FIGS. 8A-B show a report 1800 for a stroke patient with a profound impact on the patent that may also include a brain health indicator in the form of a global spectral analysis at various places in the brain. A QEEG magnitude spectra shows a frontal (peak ˜2.4 μV), central (peak ˜2.3 μV) and posterior (peak ˜2.8 μV) EEG over 0.0 to 25.0 frequency (Hz) is shown. Global spectral analysis 1810 is also shown with a current EEG and a previous EEG.

FIGS. 9A-B show a report 1900, 1910 for a 3.5 year old male presenting with high functioning autism spectrum disorder (ASD). The patient received 35 sessions of MeRT. Initially there are improvements in sleep, gradual developments in interaction and awareness. Patient is showing continued improvement in imitation, language and eye contact. Stimulation at 9.4 Hz PZ, Fpz. Note relative power alpha decreasing with increase in alpha synchrony. May occur with transition from diffuse—synchronous activities.

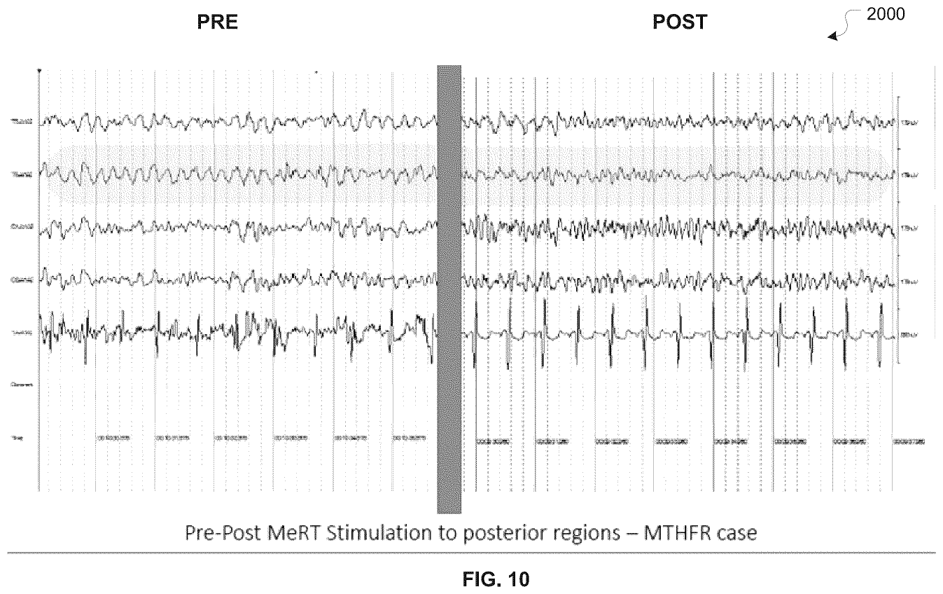

FIG. 10 show a report 2000 of pre and post stimulation (e.g., Magnetic e-Resonance Therapy (MeRTSM) which may be a treatment that may combine rTMS, qEEG, and ECG/EKG to deliver treatment for each patient's unique brain) for posterior regions of an autistic patient.

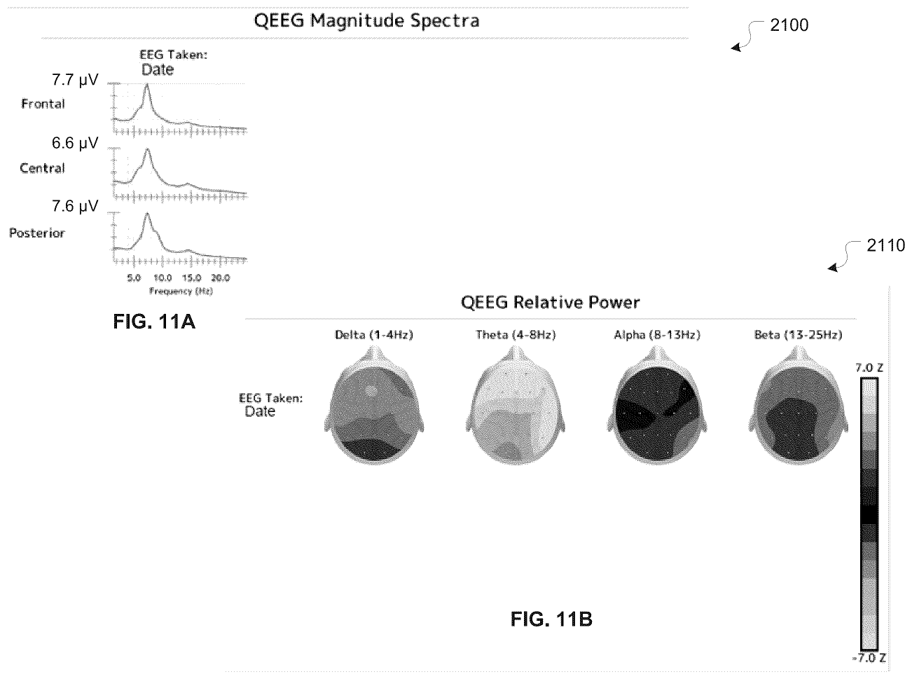

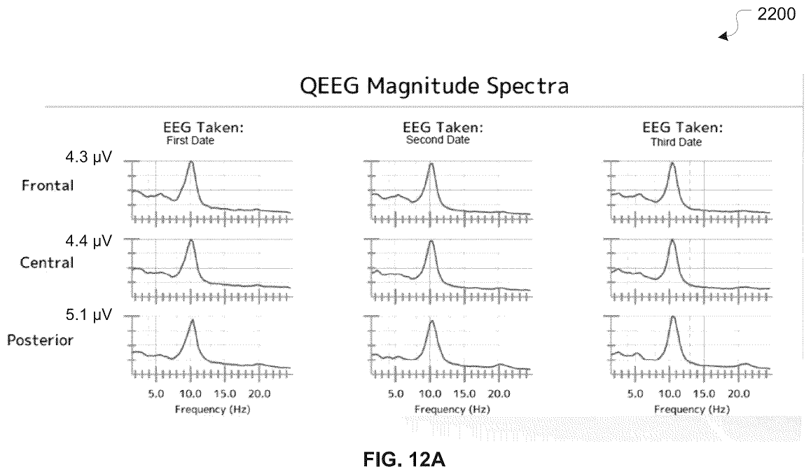

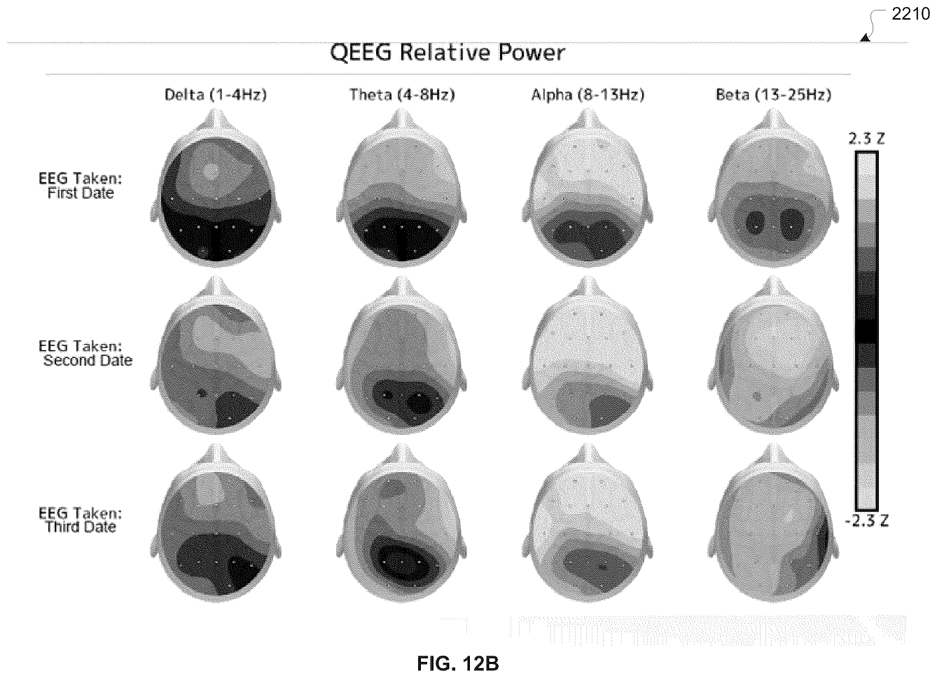

FIGS. 11A-B show a report 2100, 2110 indicating PTSD and depression for a 36 year old male with traumatic brain injury, chronic back pain, tinnitus, insomnia, obstructive sleep apnea (treated with a CPAP), hypogonadism, kidney stones, perirectal abscess with drains in place, and a suicidal attempt at a first date and a second date. Medications are: oxycontin, percocet, prazosin, prozac, cialis, hydroxyzine, and Lyrica.

FIG. 12A-B shows a report 2200, 2210 indicating anxiety and depression for a 37 year old male with 24 total MeRT sessions. There are naturally elevated beta activity, and disrupted alpha function.

Clinical Trial

202 total participants at 17 sites across the United States with a primary indication of major depression and a primary outcome of a change in Hamilton depression rating scale after 6 weeks of treatment. The intent-to-treat results: active −7.49, sham −6.97; per-protocol (remove patients with protocol violations or invalid EEG recording. Active: −9.00, sham: −6.56 (p=0.033); active −8.58, sham −4.25 (p=0.017). Very high sham response. As a comparison, neuronetics active treatment was −5.25.

Confirmatory Study

120 participants. Intent to treat results: Active −7.85, sham −6.90. Following additional 6 week open label: active −13.58, sham −12.95. Active 62.5% responders, sham 33.3% responders. Looking at subjects who had alpha frequency >9.8 Hz after just 6 week blinded phase. Active −9.82, sham −5.16 (p<0.05); active 31.6 responders, sham 12.1% responders. After 12 weeks of treatment, 62.5% were responders and 40% reached remission (HAM-D<8). Conclusion: participants with IAF >9.8 showed significantly greater improvement over sham. There is continued improvement in the open label phase, meaning longer treatment may provide to be effective.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein might be employed in practicing the invention. It is intended that the claims define the scope of the invention and that methods and structures within the scope of these claims and their equivalents be covered thereby.

1. A method for providing synchronous transcranial magnetic stimulation (TMS) comprising:
capturing electroencephalogram (EEG) data from a brain of a person;
analyzing the EEG data to identify one or more EEG characteristics relating to a brain state of the person;
determining a synchronous TMS treatment based on one or more of: the one or more EEC characteristics or the brain state; and
providing synchronous TMS to the brain of the person.

2. The method of claim 1 further comprising comparing the EEG data to modelled EEG data for the person.

3. The method of claim 1 further comprising reporting the brain state as a brain health indicator for the person based on the EEG data.

4. The method of claim 1 further comprising providing a diagnosis for the patient based on the EEG data.

5. The method of claim 1 further comprising capturing additional EEG data from the patient and determining an additional synchronous TMS treatment based on the additional EEG data.

6. The method of claim 1 further comprising providing neuromodulation based on the EEG data.

7. A device for providing transcranial magnetic stimulation (TMS) comprising:
an EEG capturing system operable to capture EEG data;
a TMS treatment system operable to communicate with an electrophysiology database to determine a synchronous TMS treatment based on the EEG data; and
a neuro-electroencephalogranm synchronization therapy (NEST) headset operable to provide the synchronous TMS treatment.

8. The device of claim 7 wherein the NEST headset is operable to produce a frequency spectrum at a concentration that is greater than a concentration produced using repetitive TMS.

9. The device of claim 7 wherein the TMS treatment system is operable to determine, based on the EEG data, an intrinsic frequency for the synchronous TMS treatment.

10. The device of claim 9 wherein the TMS treatment system is operable to determine the intrinsic frequency using one or more of a fast Fourier transform (FFT), a correlation, curve-fitting, or ringing-effect stimulation.

11. The device of claim 7 wherein the TMS treatment system is operable to determine the intrinsic frequency using one or more of a heart rate, a respiratory rate, or a gastrointestinal movement rate.

12. The device of claim 7 wherein the TMS treatment system is operable to determine the synchronous TMS treatment based on an optimal brain profile.

13. The device of claim 7 wherein the NEST headset is operable to provide neuromodulation at one or more specific frequencies having one or more specific phases at one or more specific locations.

14. The device of claim 7 wherein the NEST headset is operable to provide the synchronous TMS treatment based on a timing of one or more EEG waveforms.

15. The device of claim 7 wherein the TMS treatment system is operable to determine a brain classification used to provide the synchronous TMS treatment.

16. A method for monitoring treatment of a brain of a person, comprising:
capturing electroencephalogram data from the brain of the person;
identify a treatment type;
determine a monitoring test based on the treatment type; and
monitor a treatment effect using the monitoring test.

17. The method of claim 16 wherein the treatment type comprises one or more of a drug treatment, a device treatment, or a treatment protocol.

18. The method of claim 16 wherein the monitoring test comprises one or more of compliance testing, safety testing, efficacy testing, or biocompatibility testing.

19. The method of claim 16 wherein the monitoring test is based on a patient population type.

20. The method of claim 16 wherein the treatment comprises one or more of post-traumatic stress disorder (PTSD), attention deficit hyperactivity disorder (ADHD), or traumatic brain injury.
